(19) United States
(12) Patent Application Publication
(10) Pub. No.: US 2021/0007645 A1
(43) Pub. Date: Jan. 14, 2021

(54) BLOOD TEST SUPPORT DEVICE, BLOOD TEST SUPPORT SYSTEM, BLOOD TEST SUPPORT METHOD, AND PROGRAM

(71) Applicant: FUJIFILM Corporation, Tokyo (JP)

(72) Inventors: Haruyasu NAKATSUGAWA, Ashigarakami-gun (JP); Seiji YAMASHITA, Ashigarakami-gun (JP); Tatsuya ISHIZAKA, Ashigarakami-gun (JP)

(73) Assignee: FUJIFILM Corporation, Tokyo (JP)

(21) Appl. No.: 17/032,106

(22) Filed: Sep. 25, 2020

Related U.S. Application Data
(63) Continuation of application No. PCT/JP2019/012375, filed on Mar. 25, 2019.

(30) Foreign Application Priority Data
Mar. 28, 2018 (JP) 2018-062722

(51) Int. Cl. A61B 5/145; A61B 5/151; A61B 5/01; A61B 5/024; A61B 5/08; A61B 5/0402; A61B 5/00; G16H 40/20; G16H 10/40; G16H 50/20; G06Q 10/10; G16H 20/30; G16H 20/60; G16H 40/67; G09B 19/00

(52) U.S. Cl. CPC A61B 5/14532; A61B 2562/08; A61B 5/01; A61B 5/02405; A61B 5/0816; A61B 5/0402; A61B 5/14542; A61B 5/4806; A61B 5/486; G16H 40/20; G16H 10/40; G16H 50/20; G06Q 10/1093; G16H 20/30; G16H 20/60; G16H 40/67; G09B 19/00; A61B 2560/0252; A61B 5/151

(57) ABSTRACT

Provided are a blood test support device, a blood test support system, a blood test support method, and a program that can reduce a burden on a medical examinee and obtain highly accurate test results. The blood test support device includes an information acquisition unit that acquires an order for a blood test kit from a medical examinee; a storage unit that stores the medical examinee and an identification number given to the blood test kit in association with each other; a processing unit that calculates a recommended date and time at which blood collection is performed using the blood test kit to which the identification number is given; and an information output unit that outputs the identification number of the blood test kit and the recommended date and time in placing the order from the medical examinee.

### CROSS-REFERENCE TO RELATED APPLICATIONS

The present application is a Continuation of PCT International Application No. PCT/JP2019/012375 filed on Mar. 25, 2019 claiming priority under 35 U.S.C § 119(a) to Japanese Patent Application No. 2018-062722 filed on Mar. 28, 2018. Each of the above applications is hereby expressly incorporated by reference, in its entirety, into the present application.

### BACKGROUND OF THE INVENTION

### 1. Field of the Invention

The present invention relates to a blood test support device, a blood test support system, a blood test support method, and a program, and particularly to a recommended date and time for a blood test.

### 2. Description of the Related Art

In recent years, it has been practiced that a medical examinee who desires a test self-collects blood himself/herself and mails the collected blood sample to a medical agency or a test agency to examinee the health condition of the medical examinee. For example, JP2009-252218A discloses a self-blood collection test system that allows a medical examinee to browse test results and the like from the Internet. In JP2009-252218A, the medical examinee can browse the test results of a self-blood collection test kit, which has performed the blood collection, via a network.

### SUMMARY OF THE INVENTION

Meanwhile, a date and time at which the blood collection is performed is important in order to obtain highly accurate results in the test. However, it is a heavy burden for the medical examinee to manage a date and time for the self-blood collection.

The present invention has been made in view of such circumstances, and an object thereof is to provide a blood test support device, a blood test support system, a blood test support method, and a program that can reduce a burden on a medical examinee and obtain highly accurate test results.

A blood test support device according to a first aspect comprises an information acquisition unit that acquires an order for a blood test kit from a medical examinee; a storage unit that stores the medical examinee and an identification number given to the blood test kit in association with each other; a processing unit that calculates a recommended date and time at which blood collection is performed using the blood test kit to which the identification number is given; and an information output unit that outputs the identification number of the blood test kit and the recommended date and time in placing the order from the medical examinee.

According to the first aspect, by notifying the medical examinee of the recommended date and time at which the blood collection is performed, a burden on the medical examinee can be reduced, and a highly accurate test can be performed.

In the blood test support device according to a second aspect, the processing unit calculates the recommended date and time except a case where a blood-collection performance date or delivery period is at least one of Saturday, Sunday, a national holiday, a long holiday season, and year-end or New Year holidays.

According to the second aspect, the period from the self-blood collection of blood to the arrival of the blood test kit at the test agency can be reduced, and the blood test kit can be delivered on a testable day of the test agency.

In the blood test support device according to a third aspect, the processing unit calculates the recommended date and time on the basis of at least one of a season, an outside temperature of an area, or a presence or absence of a cooling agent, in a place of residence of the medical examinee.

According to the third aspect, the influence of the outside temperature on the blood test can be reduced.

In the blood test support device according to a fourth aspect, the information acquisition unit acquires a performance date and time for blood collection from the medical examinee, and the processing unit stores the performance date and time and the identification number in association with each other in the storage unit.

According to the fourth aspect, the performance date and time for self-blood collection can be confirmed for each blood test kit by acquiring the performance date and time for blood collection from the medical examinee.

In the blood test support device according to a fifth aspect, in a case where the processing unit determines that the performance date and time and the recommended date and time are different from each other, the information output unit outputs warning information.

According to the fifth aspect, the medical examinee can be prompted to perform the self-blood collection on the recommended date and time.

In the blood test support device according to a sixth aspect, the information acquisition unit acquires a test result of the blood test kit to which the identification number is given, and the processing unit stores the identification number, the performance date and time, and the test result in association with the medical examinee in the storage unit.

According to the sixth aspect, since the performance date and time and the identification number are stored in association with each other, a relationship between the date and time for self-blood collection and the test result can be confirmed, and this can be useful for calculating the next recommended date and time.

In the blood test support device according to a seventh aspect, in a case where the information acquisition unit acquires the order for the blood test kit from the medical examinee, the processing unit calculates the recommended date and time on the basis of the performance date and time of the medical examinee and the test result stored in the storage unit.

According to the seventh aspect, since the recommended date and time is calculated on the basis of the past test history of the medical examinee, a more accurate test result can be obtained.

In the blood test support device according to an eighth aspect, a date and time at which early morning, fasting, and rest are predicted is calculated as the recommended date and time.

According to the eighth aspect, the blood glucose level can be measured before the rise thereof.

In the blood test support device according to a ninth aspect, the processing unit calculates a self-blood collection after a meal as the recommended date and time in a case where the performance date and time is a fasting time and a blood glucose level and an HbA1c are normal.

According to the ninth aspect, a test for the postprandial hyperglycemia can be promoted, and the diabetes can be early found.

In the blood test support device according to a tenth aspect, the processing unit calculates a self-blood collection after a meal as the recommended date and time in a case where the performance date and time is only a fasting time and a blood glucose level and an HbA1c are normal, calculates the fasting time as the recommended date and time in a case where the performance date and time is not the fasting time or an unknown time, and the blood glucose level and the HbA1c are normal, calculates a performance date and time for blood collection in a case where the blood glucose level and the HbA1c are high as the recommended date and time, in a case where the performance date and time is not the fasting time or an unknown time and the blood glucose level and the HbA1c are high either at the fasting time or after the meal, and calculates the fasting time the after-the-meal as the recommended date and time in a case where the blood glucose level and the HbA1c are high both at the fasting time and after the meal and the after-the-time as the recommended date and time in a case where the performance date and time is an unknown time.

According to the tenth aspect, the probability of the diabetes can be early found.

A blood test support system according to an eleventh aspect is a blood test support system comprising a server device configured to be connectable to a terminal device of a medical examinee via a network, and the server device includes an information acquisition unit that acquires an order for a blood test kit from a medical examinee; a storage unit that stores the medical examinee and an identification number given to the blood test kit in association with each other; a processing unit that calculates a recommended date and time at which blood collection is performed using the blood test kit to which the identification number is given; and an information output unit that outputs the identification number of the blood test kit and the recommended date and time in placing the order from the medical examinee.

According to the eleventh aspect, the same effects as those of the first aspect can be obtained.

In the eleventh aspect, the same items as the items specified in the second to tenth aspects can be appropriately combined. In that case, the components that take the processing and functions specified in the blood test support device can be grasped as the components of the blood test support system that takes the processing and functions corresponding to this.

A blood test support method according to a twelfth aspect comprises an information acquisition step of acquiring an order for a blood test kit from a medical examinee; a storage step of storing the medical examinee and an identification number given to the blood test kit in association with each other; a processing step of calculating a recommended date and time at which blood collection is performed using the blood test kit to which the identification number is given; and an information output step of outputting the identification number of the blood test kit and the recommended date and time in placing the order from the medical examinee.

According to the twelfth aspect, the same effects as those of the first aspect can be obtained.

A blood test support device according to a thirteenth aspect comprises a storage unit that stores a medical examinee and a test result of the medical examinee in association with each other; a processing unit that prepares a lifestyle improvement plan for the medical examinee on the basis of the test result; an information output unit that outputs the lifestyle improvement plan to the medical examinee; and an information acquisition unit that acquires an order for a blood test kit from a medical examinee, the information acquisition unit acquires body-related information of the medical examinee and an identification number given to the blood test kit, the storage unit stores the body-related information and the identification number in association with each other, the processing unit calculates a recommended date and time at which blood collection is performed using the blood test kit to which the identification number is given on the basis of the body-related information of the medical examinee, and the information output unit outputs the identification number of the blood test kit and the recommended date and time to the medical examinee.

According to the thirteenth aspect, a burden on the medical examinee can be reduced by notifying the medical examinee of the recommended date and time at which the blood collection is performed. A relationship between the lifestyle improvement plan and the test result can be easily grasped.

In the blood test support device according to a fourteenth aspect, the lifestyle improvement plan includes information on any of diet, exercise, smoking, or supplements.

According to the fourteenth aspect, the lifestyle improvement of the medical examinee can be realized by including the diet, exercise, smoking, and supplements in the lifestyle improvement plan. In addition, the diet include food and drink.

A blood test support system according to a fifteenth aspect is a blood test support system comprising a server device configured to be connectable to a terminal device of a medical examinee via a network, the server device includes a storage unit that stores a medical examinee and a test result of the medical examinee in association with each other; a processing unit that prepares a lifestyle improvement plan for the medical examinee on the basis of the test result; an information output unit that outputs the lifestyle improvement plan to the medical examinee; and an information acquisition unit that acquires an order for a blood test kit from a medical examinee, the information acquisition unit acquires body-related information and an identification number given to the blood test kit, the storage unit stores the body-related information and the identification number in association with each other, a recommended date and time at which blood collection is performed using the blood test kit to which the identification number is given is calculated on the basis of the body-related information of the medical examinee, and the information output unit outputs the identification number of the blood test kit and the recommended date and time to the medical examinee.

According to the fifteenth aspect, the same effects as those of the thirteenth aspect can be obtained.

In the blood test support system according to a sixteenth aspect, the information acquisition unit acquires the body-related information via a terminal device owned by the medical examinee.

According to the sixteenth aspect, the body-related information can be acquired via the terminal device owned by the medical examinee. In the server device, the recommended date and time can be easily calculated.

In the blood test support system according to a seventeenth aspect, the terminal device includes at least one of a mobile terminal or a biosensor.

According to the seventeenth aspect, the body-related information can be acquired in the server device regardless of the location and time of the medical examinee.

In the blood test support system according to an eighteenth aspect, the mobile terminal includes at least one of a diet calorie calculation application program, a smoking management application program, or a drinking amount management application program.

According to the eighteenth aspect, the medical examinee can easily acquire the body-related information by using the various application programs.

In the blood test support system according to a nineteenth aspect, the biosensor is capable of acquiring information on any of an activity amount, an electrocardiogram, a heart rate, a heartbeat fluctuation, a respiratory state, a body surface temperature, a sleep analysis, a blood glucose level, or a blood oxygen concentration of the medical examinee.

According to the nineteenth aspect, the medical examinee can easily acquire the body-related information by using the biosensor.

A blood test support method according to the twentieth aspect is a blood test support method comprising a storage step of storing a medical examinee and a test result of the medical examinee in association with each other; a processing step of preparing a lifestyle improvement plan for the medical examinee on the basis of the test result; an information output step of outputting the lifestyle improvement plan to the medical examinee; and an information acquisition step of acquiring an order for a blood test kit from a medical examinee, in the information acquisition step, the body-related information of the medical examinee and an identification number given to the blood test kit are acquired, in the storage step, the body-related information and the identification number are stored in association with each other, in the processing step, a recommended date and time at which blood collection is performed using the blood test kit to which the identification number is given is calculated on the basis of the body-related information of the medical examinee, and in the information output step, the identification number of the blood test kit and the recommended date and time are output to the medical examinee.

According to the twentieth aspect, the same effect as those of the thirteenth aspect can be obtained.

A program according to a twenty-first aspect is a program for causing a computer to realize a blood test support function comprising an information acquisition function of acquiring an order for a blood test kit from a medical examinee; a storage function of storing the medical examinee and an identification number given to the blood test kit in association with each other; a processing function of calculating a recommended date and time at which blood collection is performed using the blood test kit to which an identification number is given; and an information output function of outputting the identification number of the blood test kit and the recommended date and time in placing the order from the medical examinee.

According to the twenty-first aspect, the same effects as that of the first aspects can be obtained.

A program according to a twenty-second aspect is a program for causing a computer to realize a blood test support function comprising a storage function of storing a medical examinee and a test result of the medical examinee in association with each other; a processing function of preparing a lifestyle improvement plan for the medical examinee on the basis of the test result; an information output function of outputting the lifestyle improvement plan to the medical examinee; and an information acquisition function of acquiring an order for a blood test kit from a medical examinee, in the information acquisition function, the body-related information of the medical examinee and an identification number given to the blood test kit are acquired, in the storage function, the body-related information and the identification number are stored in association with each other, In the processing function, a recommended date and time at which blood collection is performed using the blood test kit to which the identification number is given is calculated on the basis of the body-related information of the medical examinee, and in the information output function, the identification number of the blood test kit and the recommended date and time are output to the medical examinee.

According to the twenty-second aspect, the same effects as those of the thirteenth aspect can be obtained.

According to the present invention, the burden on the medical examinee can be reduced, and highly accurate test results can be obtained.

### BRIEF DESCRIPTION OF THE DRAWINGS

FIG. 1 is a diagram illustrating a schematic configuration of a blood test support system according to a first embodiment.

FIG. 2 is a block diagram illustrating a configuration example of hardware of a server device.

FIG. 3 is a functional block diagram of a server device.

FIG. 4 is a block diagram illustrating a configuration example of hardware of a terminal device.

FIG. 5 is a flowchart illustrating a procedure flow of a blood test support method according to the first embodiment.

FIG. 6 is a diagram illustrating a flow of information and a blood test kit in the blood test support system according to the first embodiment.

FIG. 7 is a diagram illustrating a flow of recommended date-and-time calculation processing.

FIG. 8 is a diagram illustrating an example of a recommended date-and-time display screen.

FIG. 9 is a diagram illustrating another example of the recommended date-and-time display screen.

FIG. 10 is a diagram illustrating still another example of the recommended date-and-time display screen.

FIG. 11 is a diagram illustrating a schematic configuration of a blood test support system of a second embodiment.

FIG. 12 is a functional block diagram of a server device.

FIG. 13 is a flowchart illustrating a procedure flow of a blood test support method according to the second embodiment.

FIG. 14 is a diagram illustrating a flow of information and a blood test kit and a biosensor in the blood test support system according to the second embodiment.

FIG. 15 is a diagram illustrating an example of a screen for approving a lifestyle improvement plan.

### DESCRIPTION OF THE PREFERRED EMBODIMENTS

Hereinafter, preferred embodiments of the present invention will be described with reference to the accompanying drawings. The present invention will be described by the following preferred embodiments. Modifications can be made by many techniques and other embodiments than the embodiments can be utilized, without departing from the scope of the present invention. Therefore, all modifications within the scope of the present invention are included in the claims. In the present specification, in a case where the numerical range is expressed using “from”, the numerical values of an upper limit and a lower limit indicated by “from” are included in the numerical range. In the present specification, regarding information, signals, and the like, output and transmission can be replaced with each other, and acquisition and reception can be replaced with each other.

### First Embodiment

[Overall Configuration of Blood Test Support System]

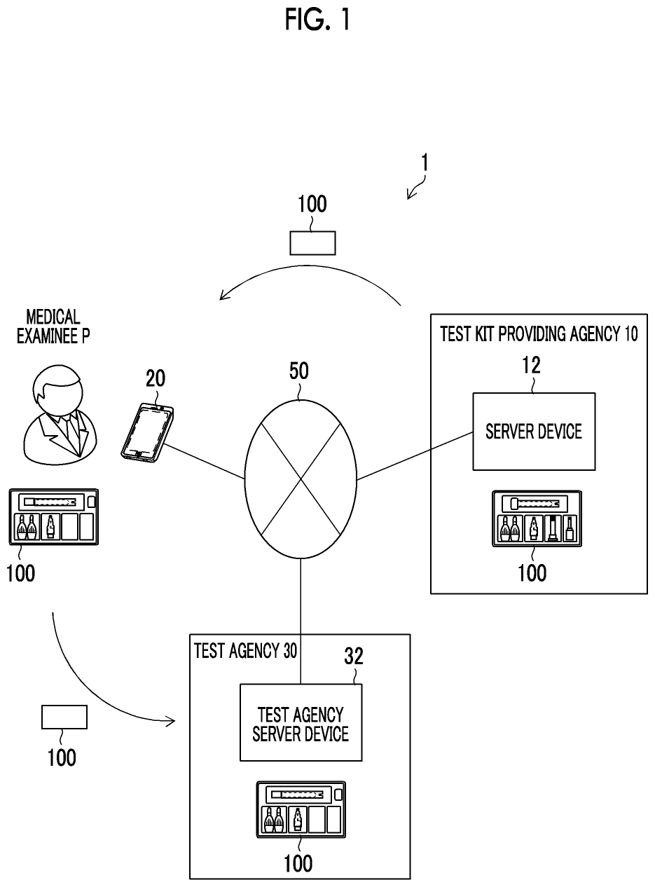

FIG. 1 is a diagram illustrating a configuration example of the blood test support system of the first embodiment. As illustrated in FIG. 1, a blood test support system 1 comprises a server device 12 of a test kit providing agency 10 that is connected such that data communication is possible via a network 50, and a terminal device 20 owned by a medical examinee P. In addition, the server device 12 is equivalent to an example of a blood test support device.

In the embodiment illustrated in FIG. 1, the server device 12 is connected to a test agency server device 32 of a test agency 30 via the network 50.

Upon receiving a request from the medical examinee P, the test kit providing agency 10 provides the medical examinee P with a blood test kit 100 capable of performing self-blood collection. Specific examples of the blood test kit 100 include a biological sample separation instrument described in the specification of Japanese Patent No. 3597827, a sample analysis tool described in the specification of Japanese Patent No. 3643011, and the like. Moreover, it is possible to include treatment tools such as a lancet, a plaster, and a sterile cloth, and an instruction manual used in a case where blood is collected. In addition, each blood test kit 100 is given a unique identification number.

The medical examinee P is a person who desires to acquire a test result by using the blood test kit 100, and is also referred to as a subject. The medical examinee P can order the blood test kit 100 from the terminal device 20 to the test kit providing agency 10 via the network 50. The terminal device 20 may be, for example, a mobile terminal represented by a smartphone, a tablet, or the like or may be a personal computer.

The medical examinee P receives the blood test kit 100 sent from the test kit providing agency 10. The medical examinee P collects blood using the blood test kit 100. The medical examinee P sends the blood test kit 100 for which blood collection has been completed to the test agency 30.

The test agency 30 receives the blood test kit 100 sent from the medical examinee P. The test agency 30 tests the collected blood sample of the blood test kit 100 using a blood analysis device or the like. The test agency 30 acquires a numerical value for each test item for the blood sample. The test agency 30 can store the test result in the test agency server device 32 in association with the identification number of the blood test kit 100.

The test kit providing agency 10 can acquire the test result stored in the test agency server device 32 into the server device 12 via the network 50 and store the test result in association with the identification number of the blood test kit 100.

In FIG. 1, the test kit providing agency 10 and the test agency 30 are separate agencies, but may be an agency in which the test kit providing agency 10 and the test agency 30 are integrated with each other. In the case of the integrated agency, the server device 12 can comprise the function of the test agency server device 32.

Generally, the date and time at which blood collection is performed is entrusted to the management of the medical examinee P. However, there is a case where an accurate test result is not obtained depending on the date and time at which blood collection is performed. For example, since the blood glucose level and the cholesterol level rise after a meal, the after-a-meal is generally not preferable as a date and time at which self-blood collection is performed in a case where highly accurate test results are to be obtained.

On the other hand, it is also contemplated that lifestyle is further improved by testing a self-collected blood sample after a meal from a test result of the self-blood collection performed in the past. It is a heavy burden for the medical examinee P to manage the performance date and time at which blood collection is performed. Additionally, it is not easy for the medical examinee P to determine a performance date and time at which highly accurate results can be obtained from a past test result.

In the blood test support system 1 of the first embodiment illustrated in FIG. 1, it is possible to acquire a highly accurate test result by transmitting a fasting time to the terminal device 20 of the medical examinee P as the recommended date and time at which the self-blood collection is performed. Additionally, in a case where there is a past test result, not only the fasting time but also the after-a-meal or the like are transmitted to the terminal device 20 of the medical examinee P as the recommended date and time at which the self-blood collection is performed, so that the presence or absence of postprandial hyperglycemia, which is an initial symptom of diabetes, is transmitted by transmitting, can be tested, which can lead to early detection of diabetes. In the present specification, there is a case where the self-blood collection is simply referred to as blood collection.

The postprandial hyperglycemia is a symptom observed in the early stage of diabetes, and means a symptom in which the blood glucose level becomes high about 1 to 2 hours after a meal even in a case where the fasting blood glucose level is within the normal range. The postprandial hyperglycemia has a problem that, in a case where the postprandial hyperglycemia is left untreated, this may progress to diabetes and the risk of cerebral infarction, myocardial infarction, or the like increases. The possibility of the postprandial hyperglycemia can be known by confirming the value of HbA1c by a blood test. In a case where the value of HbA1c exceeds 6.5%, the possibility of diabetes is high. Therefore, even in a case where the fasting blood glucose level is low, it is preferable to take the glucose tolerance test in a case where the value of HbA1c exceeds 6.5%.

The configuration of the blood test support system 1 of the first embodiment will be described.

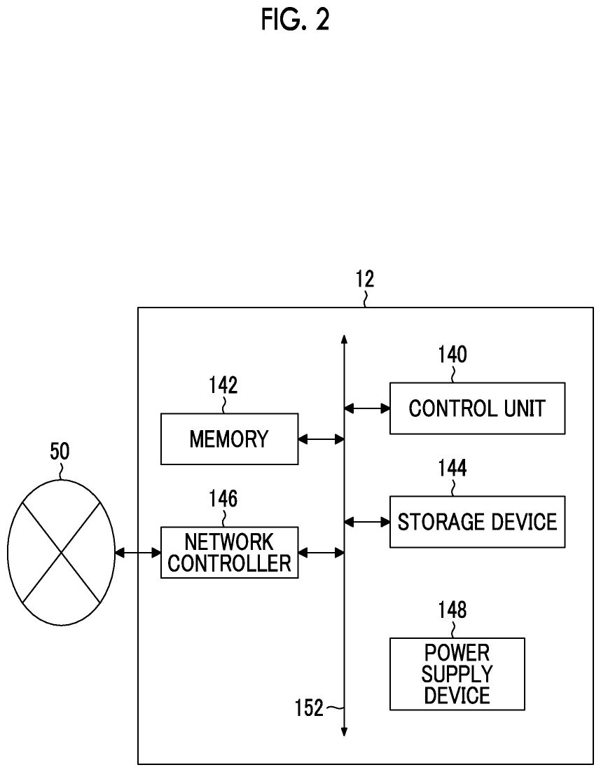

FIG. 2 is a diagram illustrating a configuration example of hardware of the server device 12. The server device 12 comprises a control unit 140, a memory 142, a storage device 144, a network controller 146, and a power supply device 148. The control unit 140, the memory 142, the storage device 144, and the network controller 146 are connected to each other via a bus 152 so that data communication is possible.

The control unit 140 functions as an overall control unit, various calculation units, and a storage control unit of the server device 12. The control unit 140 executes a program stored in a read only memory (ROM) included in the memory 142.

The control unit 140 downloads a program from an external storage device via the network controller 146, and executes the downloaded program. The external storage device may be communicably connected to the server device 12 via the network 50.

The control unit 140 uses a random access memory (RAM) included in the memory 142 as a calculation region, and executes various kinds of processing in cooperation with various programs. As a result, various functions of the server device 12 are realized.

The control unit 140 controls the reading of data from the storage device 144 and the writing of data to the storage device 144. The control unit 140 may acquire various data from the external storage device via the network controller 146. The control unit 140 can execute various kinds of processing such as calculation using the acquired various data.

The control unit 140 may include one or more processors. Examples of the processor include a (field programmable gate array (FPGA) and a programmable logic device (PLD). The FPGA and the PLD allow the circuit configuration to be changed after manufacture.

Another example of the processor is an application specific integrated circuit (ASIC). The ASIC comprises a circuit configuration exclusively designed to execute specific processing.

The control unit 140 can apply the same type of two or more processors. For example, the control unit 140 may use two or more FPGAs or two PLDs. The control unit 140 may apply different types of two or more processors. For example, the control unit 140 may apply one or more FPGAs and one or more ASICs.

In a case where a plurality of the control units are provided, the plurality of control units may be configured using one processor. As an example of configuring the plurality of control units with one processor, there is a form in which one processor is configured using a combination of one or more central processing units (CPUs) and software, and the processor functions as the plurality of control units. Instead of the CPU or in combination with the CPU, a graphics processing unit (GPU), which is a processor specialized in image processing, may be applied. In addition, the software referred to herein is synonymous with a program. A representative example in which the plurality of control units are configured using one processor is a computer such as a client device and a server device.

As another example in which the plurality of control units are configured by one processor, there is a form in which a processor that realizes the functions of an entire system including the plurality of control units by one IC chip is used. A representative example of the processor that realizes the functions of the entire system including the plurality of control units by one IC chip is a system on chip (SoC). In addition, IC is an abbreviation for Integrated Circuit. In this way, the control unit 140 is configured using one or more of various processors as a hardware structure.

The memory 142 comprises a ROM (not illustrated) and a RAM (not illustrated). The ROM stores various programs executed by the server device 12. The ROM stores parameters used for executing the various programs, files, and the like. The RAM functions as a temporary storage region for data, a work region for the control unit 140, and the like.

The storage device 144 stores various data non-temporarily. The storage device 144 may be externally attached to the outside of the server device 12. A large-capacity semiconductor memory device may be applied instead of or in combination with the storage device 144.

The network controller 146 controls data communication between the terminal device 20 illustrated in FIG. 1 and external devices such as the test agency server device 32. The control of the data communication may include management of traffic for the data communication.

As the power supply device 148, a large-capacity power supply device such as an uninterruptible power supply (UPS) is applied. The power supply device 148 supplies power to the server device 12 in a case where a commercial power source is shut down due to a power failure or the like. In addition, the hardware configuration of the server device 12 illustrated in FIG. 2 is an example, and addition, deletion, and change can be appropriately made.

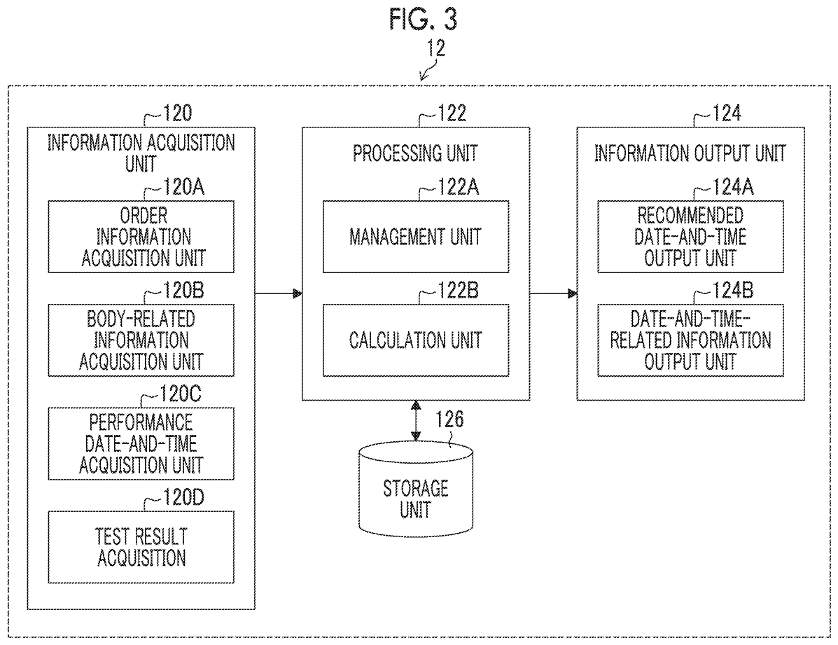

FIG. 3 is a functional block diagram of the server device 12. The server device 12 illustrated in FIG. 3 comprises an information acquisition unit 120, a processing unit 122, an information output unit 124, and a storage unit 126. The details of the respective units will be described below.

The information acquisition unit 120 comprises an order information acquisition unit 120A that acquires an order from the medical examinee P, a body-related information acquisition unit 120B, a performance date-and-time acquisition unit 120C, and a test result acquisition unit 120D. The information acquisition unit 120 may comprise various information acquisition units that acquire various information. Illustration of the various information acquisition units is omitted.

The order information acquisition unit 120A acquires personal information such as the name, birth date, address, telephone number, e-mail address, and payment method of the medical examinee P. In a case where the medical examinee P is a medical examinee whom a test using the blood test kit 100 has been performed in the past, the order information acquisition unit 120A can acquires information such as an identification number, a password, and the like for identifying the medical examinee P.

The body-related information acquisition unit 120B acquires the body-related information of the medical examinee P. The body-related information includes information obtained by measurement, information reported by the medical examinee P, and information obtained by using various management application programs. The body-related information is measured by an installed measuring device, a wearable measuring device such as a biosensor that the medical examinee P can wear, or the like. Examples of the body-related information include biological information such as height, weight, blood glucose level, and heart rate, and behavior information such as the number of exercises, the number of cigarettes smoked, the amount of alcohol drunk, the type and acquisition frequency of supplements, and meals. However, the type and acquisition method of the body-related information are not particularly limited.

The performance date-and-time acquisition unit 120C acquires the performance date and time at which the medical examinee P uses the blood test kit 100 to perform self-blood collection. The performance date and time can be acquired via the terminal device 20 of the medical examinee P. For example, the date and time at which the medical examinee P has read the identification number given to the blood test kit 100 by the terminal device 20 can be set as the performance date and time. Additionally, the date and time input to the terminal device 20 by the medical examinee P can be used as the performance date and time. In the embodiment, the terminal device 20 of the medical examinee P illustrated in FIG. 1 can input the order information, the body-related information, and the performance date and time to the information acquisition unit 120.

The test result acquisition unit 120D acquires the test result of the medical examinee P stored in the test agency server device 32 of the test agency 30 in conjunction with the identification number of the blood test kit 100. Test results include the blood glucose level, HbA1c, HDL cholesterol, LDL cholesterol, neutral fat, AST (GOT), ALT (GPT), γ-GPT, ALB, ALP, e-GFR, and the like, which are tested by blood test.

HbA1c is a glycated protein of glycohemoglobin in which glucose is bound to the N-terminal of the β chain of hemoglobin, and is sometimes referred to as glycated hemoglobin. HDL is an abbreviation for high density lipoprotein, which represents high density lipoprotein. LDL is an abbreviation for low density lipoprotein, which represents low density lipoprotein.

AST represents aspartate aminotransferase. GOT represents glutamate oxaloacetate transaminase. ALT represents alanine aminotransferase. GPT represents glutamate pyruvate transaminase. γ-GPT represents gamma glutamyl transpeptidase. ALB represents albumin. ALP represents alkaline phosphatase. e-GFR represents the estimated glomerular filtration rate.

The processing unit 122 acquires various information from the information acquisition unit 120. The processing unit 122 comprises a management unit 122A and a calculation unit 122B. The management unit 122A performs processing (for example, input, output, search, and the like) of the information acquired from the information acquisition unit 120. The management unit 122A processes information regarding the medical examinee P in association with the identification number given to the blood test kit 100. For example, the management unit 122A causes the storage unit 126 to store the information from the information acquisition unit 120 in association with the identification number, or reads the information from the storage unit 126. As a result, various information can be provided to the medical examinee P in relation to the identification number.

According to the instruction from the management unit 122A, the calculation unit 122B calculates, for each medical examinee P, a recommended date and time suitable for self-collecting blood by the blood test kit 100, on the basis of the information related to the medical examinee P. The calculation unit 122B calculates in consideration of past test results of the medical examinee P, lifestyle improvement information, climate of the medical examinee P's place of residence, postal circumstances, and the like. The calculation unit 122B can calculate a plurality of recommended dates and times for the medical examinee P.

The management unit 122A stores the recommended date and time for the self-blood collection that is calculated by the calculation unit 122B in the storage unit 126 in association with the identification number. The management unit 122A manages the recommended date and time. In the recommended date and time management, the arrival of the recommended date and time, the passage of the recommended date and time, and whether or not the performance date and time obtained from the information acquisition unit 120 and the recommended date and time match each other are managed. In a case where the recommended date and time have passed, the management unit 122A causes the calculation unit 122B to calculate the recommended date and time again.

The information output unit 124 comprises a recommended date-and-time output unit 124A and a date-and-time-related information output unit 124B. The recommended date-and-time output unit 124A outputs the recommended date and time calculated by the calculation unit 122B.

The date-and-time-related information output unit 124B outputs information related to the recommended date and time managed by the management unit 122A. The date-and-time-related information includes notification of arrival of the recommended date and time and notification of passage of the recommended date and time. Additionally, in a case where the performance date and time and the recommended date and time do not match each other, the date-and-time-related information includes warning information.

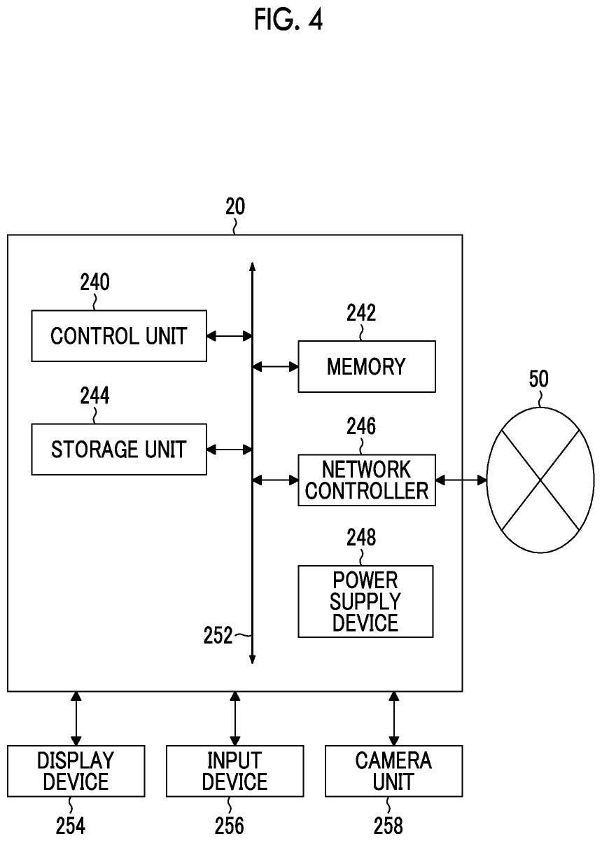

FIG. 4 is a diagram illustrating a configuration example of hardware of the terminal device 20. The terminal device 20 comprises a control unit 240, a memory 242, a storage device 244, a network controller 246, and a power supply device 248. The control unit 240, the memory 242, the storage device 244, and the network controller 246 are connected to each other via a bus 252 so that data communication is possible. The terminal device 20 comprises the same configuration as the server device 12. As the power supply device 248 of the terminal device 20, a rechargeable power source such as a lithium ion battery is used.

The terminal device 20 further comprises a display device 254, an input device 256, and a camera unit 258. The display device 254 is, for example, a liquid crystal display, and can display various information. The input device 256 is, for example, a touch panel, a keyboard, a mouse, and the like, and the medical examinee P can input and perform an instruction. The camera unit 258 includes an imaging element, a lens, and the like, captures images (still images and motion pictures), and the captured images are recorded in the storage device 244. The imaging element includes, for example, a charge coupled device (CCD), a complementary metal-oxide-semiconductor (CMOS), or the like.

In addition, the terminal device 20 may be a personal computer or a portable communication device such as a smartphone. The terminal device 20 is preferably a portable communication device. The medical examinee P can transmit various information to the server device 12 or receive various information from the server device 12, regardless of location.

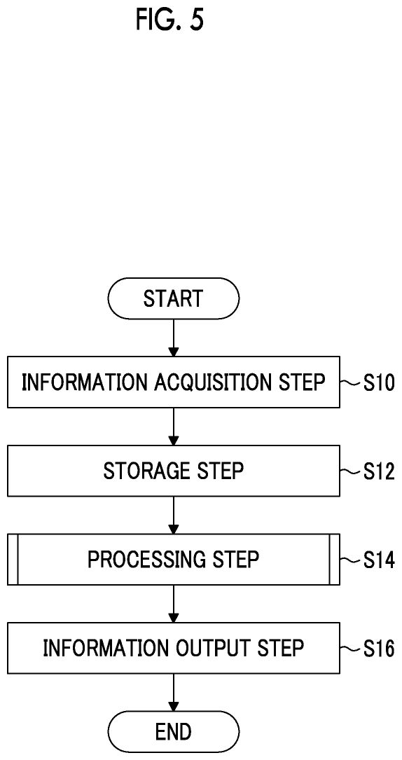

Next, a flow of a blood test support method of the first embodiment will be described. As illustrated in FIG. 5, the blood test support method comprises an information acquisition step (Step S10) of acquiring the order of the blood test kit 100 from the medical examinee P, a storage step (Step S12) of associating and storing the identification numbers given to the medical examinee P and the blood test kit 100 each other, a processing step (Step S14) of calculating a recommended date and time at which blood collection is performed using the blood test kit 100 to which the identification number is given, and an information output step (Step S16) of outputting the identification number of the blood test kit 100 and the recommended date and time in a case where the order is made from the medical examinee P.

The above-described blood test support method can be configured as a program that causes a computer to realize functions corresponding to the respective units in the blood test support device and functions corresponding to the respective steps in the blood test support method.

For example, a blood test support program that causes a computer to realize an information acquisition function, a storage function, a processing function, and an information output function may be configured.

It is possible to store the program of causing the program to realize the above-described blood test support functions in a computer-readable information storage medium that is a non-temporary information storage medium that is a tangible object, and provide the program through the information storage medium.

Additionally, instead of storing the program in the non-temporary information storage medium to provide the program, an aspect in which the program signal is provided via a network is also possible.

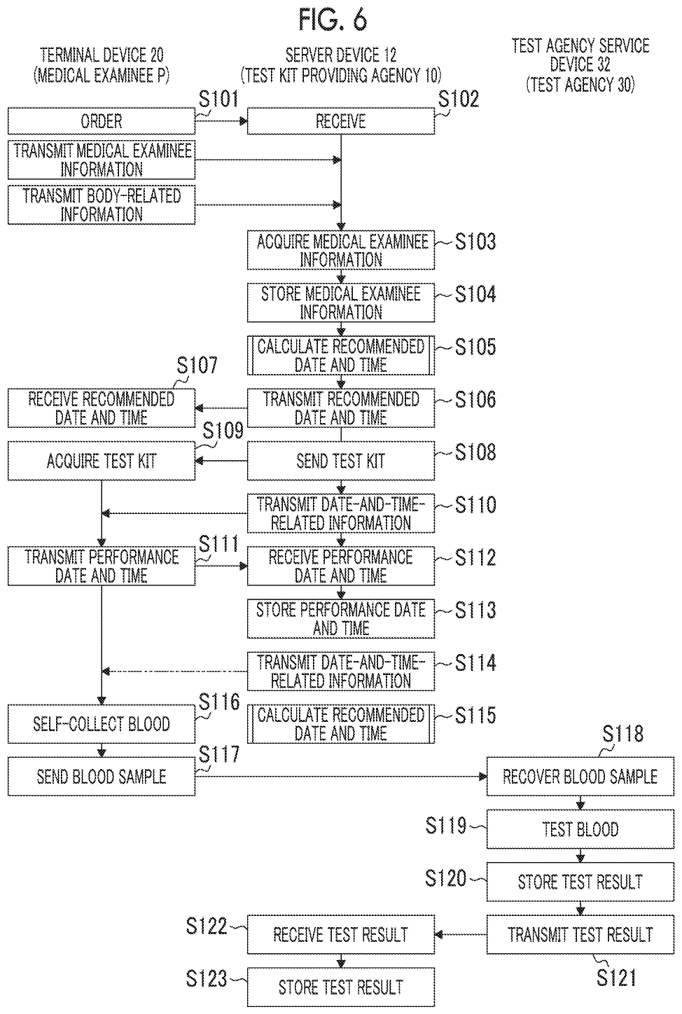

FIG. 6 is a diagram illustrating the flow of information and the flow of the blood test kit in the blood support system of the first embodiment.

In an order step S101, the order is transmitted from the terminal device 20 of the medical examinee P to the server device 12 of the test kit providing agency 10. The medical examinee P performs operations necessary for the order from the input device 256 of the terminal device 20. The order means an indication of intention of the medical examinee P who desires a blood test by the blood test kit 100. The terminal device 20 of the medical examinee P transmits the medical examinee information to the server device 12. The medical examinee information includes personal information such as the name, the birth date, and the address, of the medical examinee P and the payment method. In a case where the medical examinee P is the medical examinee P who has undergone the test in the past, there is a case where the order information includes an identification number and a password for identifying the medical examinee P. In a reception step S102, the server device 12 receives an order for a blood test using the blood test kit 100 transmitted from the terminal device 20. In the order step S101, the order information can be transmitted from the terminal device 20 to the server device 12 regarding the body-related information of the medical examinee P.

In a medical examinee information acquisition step S103, the information of the medical examinee P who ordered the blood test kit 100 is acquired. The order information is acquired via the order information acquisition unit 120A of the information acquisition unit 120. The body-related information is acquired as the medical examinee information via the body-related information acquisition unit 120B as necessary.

In a medical examinee information storage step S104, the information obtained in the medical examinee information acquisition step S103 is stored in the server device 12. The management unit 122A of the processing unit 122 stores the medical examinee P and the identification number of the blood test kit 100 in the storage unit 126 in association with each other. In the medical examinee information storage step S104, the order information and the body-related information of the medical examinee P and the identification number of the blood test kit 100 are stored in the storage unit 126 in association with each other.

After the medical examinee information storage step S104, the process proceeds to the recommended date-and-time calculation step S105. In a recommended date-and-time calculation step S105, the calculation unit 122B of the processing unit 122 calculates the recommended date and time on the basis of the order information of the medical examinee P and, as necessary, the body-related information.

In a recommended date-and-time transmission step S106, the server device 12 transmits the recommended date and time and the identification number of the blood test kit 100 to the terminal device 20. In the recommended date-and-time transmission step S106, the recommended date and time is transmitted to the terminal device 20 of the medical examinee P via the recommended date-and-time output unit 124A. The management unit 122A may transmit information, indicating that the recommended date and time has been calculated, to the terminal device 20 via the information output unit 124. For example, the management unit 122A can transmit information, indicating that the recommended date and time has been calculated, to the terminal device 20 using an e-mail, an alert, or the like.

In a recommended date-and-time reception step S107, the terminal device 20 receives the recommended date and time transmitted from the server device 12. The medical examinee P can confirm the recommended date and time via the display device 254 of the terminal device 20.

In a test kit sending step S108, the test kit providing agency 10 sends the blood test kit 100 to the medical examinee P on the basis of the order information. In a test kit acquisition step S109, the medical examinee P acquires the blood test kit sent from the test kit providing agency 10.

In a date-and-time-related information transmission step S110, the management unit 122A transmits information related to the recommended date and time to the terminal device 20 of the medical examinee P via the date-and-time-related information output unit 124B. The arrival of the recommended date and time, the passage of the recommended date and time, or the change of the recommended date and time are notified of using an e-mail, an alert, and the like.

In a performance date-and-time transmission step S111, the performance date and time is transmitted from the terminal device 20 of the medical examinee P to the server device 12. The medical examinee P can input the performance date and time from the input device 256 of the terminal device 20. The medical examinee P can input the identification number of the blood test kit 100 by the camera unit 258 of the terminal device 20 and input the imaged date and time to the terminal device 20 as the performance date and time.

In a performance date-and-time reception step S112, the server device 12 receives the performance date and time transmitted from the terminal device 20. The server device 12 acquires the performance date and time via the performance date-and-time acquisition unit 120C of the information acquisition unit 120. In a performance date-and-time storage step S113, the management unit 122A stores the performance date and time in the storage unit 126 in association with the identification number of blood test kit 100.

In addition, in a case where the performance date and time and the recommended date and time are different from each other, in a date-and-time-related information transmission step S114, the management unit 112A transmits the warning information to the terminal device 20 of the medical examinee P via the date-and-time-related information output unit 124B. In a case where the performance date and time and the recommended date and time match each other, the date-and-time-related information transmission step S114 is not performed, and the warning information is not transmitted to the terminal device 20 of the medical examinee P.

As necessary, there is a case where a recommended date-and-time calculation step S115 is executed to calculate a new recommended date and time. In a case where the recommended date-and-time calculation step S115 is executed, a recommended date-and-time transmission step and a recommended date-and-time reception step (not illustrated) are executed. The management unit 122A manages the new recommended date and time.

In a self-blood collecting step S116, the medical examinee P performs self-blood collection using the blood test kit 100 according to the recommended date and time. In a blood sample sending step S117, the medical examinee P sends the blood test kit 100, in which a blood sample is collected, to the test agency 30.

In a blood sample recovering step S118, the test agency 30 recovers the blood test kit 100 including the blood sample of the medical examinee P sent from the medical examinee P. In a blood testing step S119, the test agency 30 tests the blood sample of the medical examinee P. In a test result storage step S120, the test result is stored in a storage unit of the test agency server device 32 in association with the medical examinee P and the identification number of the blood test kit 100. In a test result transmission step S121, the blood test of the medical examinee P stored in the test agency server device 32 is transmitted to the server device 12.

In a test result reception step S122, the test result stored in the test agency server device 32 is acquired. The test result is acquired via the test result acquisition unit 120D of the information acquisition unit 120. The test result is associated with the identification number of blood test kit 100. In the test result storage step S123, the test result obtained in the test result reception step S122 is stored in the server device 12. The management unit 122A of the processing unit 122 stores the test result and the identification number of the blood test kit 100 in the storage unit 126 in association with each other.

The management unit 122A may transmit information, indicating that the test result is stored in the server device 12, to the terminal device 20 via the information output unit 124. For example, the management unit 122A can transmit the information, indicating that the test result is stored in the server device 12, to the terminal device 20, using an e-mail, an alert, or the like. In a case where the test result is stored in the server device 12, the medical examinee P can browse the test result via the terminal device 20.

In the first embodiment, since the medical examinee P is notified of a recommended date and time for self-collecting blood, highly accurate test results can be expected. Additionally, by managing the recommended date and time and notifying the medical examinee P of the information related to the recommended date and time, it is possible to prompt the performance of the self-blood collection at the recommended date and time.

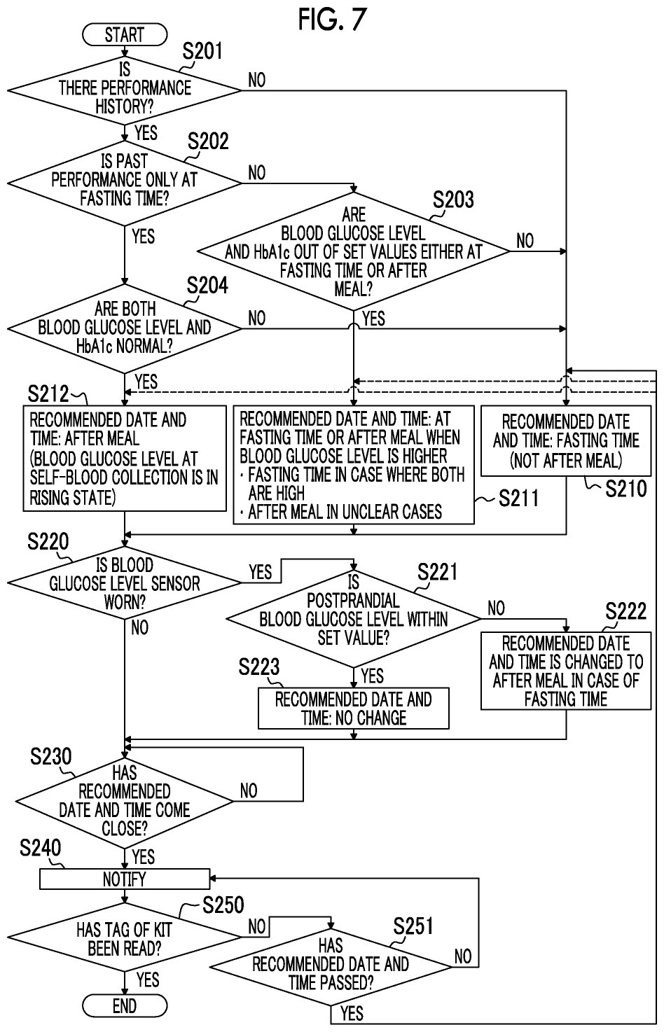

Next, the calculation of the recommended date and time according to the first embodiment will be described. FIG. 7 is a diagram illustrating an example of the flow of processing for calculating the recommended date and time.

First, in Step S201, in a case where the order information is acquired from the medical examinee P, it is determined whether or not there is a performance history. The management unit 122A searches the storage unit 126 for the test result of the medical examinee P on the basis of the order information of the medical examinee P. The calculation unit 122B calculates the recommended date and time on the basis of the search result of the management unit 122A.

As a result of Step S201, in a case where the test result of the medical examinee P is found, YES determination is made, and the calculation unit 122B proceeds with the processing to Step S202. As a result of Step S201, in a case where the test result of the medical examinee P is not found, a NO determination is made, and the calculation unit 122B proceeds with the processing to Step S210.

In Step S202, it is determined “whether or not the past performance is made only at a fasting time”. As a result of Step S202, in a case where the past performance is made only at a fasting time, a YES determination is made, and the calculation unit 122B proceeds with the processing to Step S204. In Step S202, a NO determination is made except for the YES determination, and the calculation unit 122B proceeds with the processing to Step S203.

In Step S203, it is determined “whether the blood glucose level and HbA1c are out of set values either at a fasting time or after a meal”. As a result of Step S203, in a case where the blood glucose level and HbA1c are higher than the set values, a YES determination is made, and the calculation unit 122B proceeds with the processing to Step S211. In Step S203, a NO determination is made except for the YES determination, and the calculation unit 122B proceeds with the processing to Step S210.

In Step S204, it is determined whether “the blood glucose level and the HbA1c are both normal”. As a result of Step S204, in a case where the blood glucose level and HbA1c are normal, a YES determination is made, and the calculation unit 122B proceeds with the processing to Step S212. In Step S204, a NO determination is made except for the YES determination, and the calculation unit 122B proceeds with the processing to Step S210. As for the determination of normality, cases where the blood glucose level and HbA1c are within predetermined ranges are determined to be normal. As the range of the blood glucose level, for example, a range of 80 mg/dL to 112 mg/dL can be set in advance. Additionally, as the range of HbA1c, for example, a range of 4.3% to 5.8% can be set in advance. However, the blood glucose level range and the HbA1c range are examples, and can be changed.

In the processing of Step S210, the calculation unit 122B calculates a fasting time (not after-a-meal), an early morning, and the date and time at which rest is predicted, as the recommended date and time. In a case where the medical examinee P receives the blood test for the first time, the recommended date and time is basically a fasting time. Additionally, in a case where the medical examinee P is either a diabetic person or a person who does not have postprandial hyperglycemia, the recommended date and time is basically a fasting time.

In the processing of Step S211, the calculation unit 122B calculates, as the recommended date and time, either a fasting time or after-a-meal at which the blood glucose level is higher. In a case where either the blood glucose level is higher either at a fasting time or after a meal, the calculation unit 122B calculates the fasting time as the recommended date and time. In unclear cases, the calculation unit 122B calculates the after-a-meal as the recommended date and time.

In the processing of Step S212, the calculation unit 122B calculates, as recommended date and time, after a meal (a state in which the blood glucose level at the time of the self-blood collection rises). This is because the medical examinee P has the possibility of postprandial hyperglycemia.

After the recommended date and time is calculated by any of Step S210, Step S211, or Step S212, the calculation unit 122B proceeds with the processing to Step S220.

Next, a procedure of processing in a case where the medical examinee P wears the biosensor will be described. For example, in a case where the medical examinee P orders the blood test kit 100, and the biosensor can be ordered as an option. The biosensor is, for example, a disposable wearable device. The biosensor can acquire the biological information and the behavior information, and the biological information and the behavior information include activity amount, electrocardiogram, heart rate, heartbeat fluctuation, respiratory state, body surface temperature, sleep analysis, blood glucose level, the number of steps, travel distance, the number of calories burned, blood oxygen concentration, and the like. However, the biosensor is not limited to these.

The information of the biosensor can be transmitted as the body-related information to the server device 12 via the terminal device 20 such as the mobile phone of the medical examinee P. The body-related information of the medical examinee P is acquired by the body-related information acquisition unit 120B and stored in the storage unit 126 in the management unit 122A.

By monitoring the blood glucose level with the biosensor, the result can be reflected in the calculation of the recommended date and time.

For example, in a case where there is a history of blood tests performed in the past and HbA1c (which indicates the average state of blood glucose in the past 1 to 2 months) is slightly higher than the blood glucose level, it is preferable to wear a biosensor that measures the blood glucose level. The biosensor may be disposable. By measuring the blood glucose level, the recommended date and time suitable for the medical examinee P can be calculated and transmitted to the terminal device 20 of the medical examinee P.

Additionally, for example, in a case where a biosensor capable of acquiring the activity amount is worn and the server device 12 acquires the information on the activity amount indicating a vigorous exercise from the biosensor, the calculation unit 122B calculates the recommended date and time, avoiding a few days after the exercise in consideration of an increase in GOT.

In FIG. 7, a biosensor capable of measuring the blood glucose level will be described as an example. In Step S220, it is determined “whether or not a blood glucose level sensor is worn”. As a result of the Step S220, in a case where the blood glucose level sensor is not worn, a NO determination is made, and the calculation unit 122B proceeds with the processing to Step S230. In the case of the NO determination, the recommended date and time calculated by any of Step S210, Step S211, or Step S212 is maintained without being changed.

As a result of Step S220, in a case where the blood glucose level sensor is worn, a YES determination is made, and the calculation unit 122B proceeds with the processing to Step S221.

In Step S221, it is determined “whether the postprandial blood glucose level is within a set value”. As a result of Step S221, in a case where the postprandial blood glucose level is within a set value, a YES determination is made, and the calculation unit 122B proceeds with the processing to Step S223. In Step S223, the calculation unit 122B does not change the recommended date and time, and the recommended date and time is calculated by any of Step S210, Step S211, or Step S212. Thereafter, the calculation unit 122B proceeds with the processing to Step S230.

As a result of Step S221, in a case where the postprandial blood glucose level is not within the set value, a NO determination is made, and the calculation unit 122B proceeds with the processing to Step S222. In Step S222, the calculation unit 122B calculates the after-a-meal in a case where the recommended date and time is a fasting time, and changes the recommended date and time. Thereafter, the calculation unit 122B proceeds with the processing to Step S230. In Step S222, in a case where there is a suspicion of the postprandial hyperglycemia with the blood glucose level sensor, it is preferable to calculate the after-a-meal as the recommended date and time, not at the fasting time, regardless of the presence or absence of the past performance history.

In Step S230, it is determined “whether or not the recommended date and time has come close”. As a result of Step S230, in a case where the recommended date and time have not come close, a NO determination is made, and the management unit 122A repeats Step S230 until a YES determination is made. As a result of Step S230, in a case where the recommended date and time is approaching, a YES determination is made, and the management unit 122A proceeds with the processing to Step S240. Setting of the criteria for determining whether or not the recommended date and time is approaching can be appropriately changed. For example, one hour before, two hours before, or the like of the recommended date and time can be set.

In Step S240, the date-and-time-related information indicating that the recommended date and time is approaching is output from the date-and-time-related information output unit 124B to the terminal device 20 of the medical examinee P. Thereafter, the management unit 122A proceeds with the processing to Step S250. As the date-and-time-related information, for example, it is preferable to output a display such as “will be a recommended date and time soon” to the terminal device 20 of the medical examinee P.

In Step S250, it is determined “whether or not a tag of the kit has been read”. As a result of Step S250, in a case where the tag of the blood test kit 100 is not read, a NO determination is made, and the management unit 122A proceeds with the processing to Step S251. As a result of Step S250, in a case where the tag of the blood test kit 100 is read, a YES determination is made, and the management unit 122A ends the processing. The management unit 122A stores the date and time at which the tag of the blood test kit 100 is read, in the storage unit 126 in association with the identification number of the blood test kit 100, as the performance date and time.

In Step S251, it is determined “whether or not the recommended date and time has passed. As a result of Step S251, in a case where the recommended date and time has not passed, a NO determination is made, and the management unit 122A returns the processing to Step S240. As a result of Step S251, in a case where the recommended date and time has passed, a YES determination is made, and the management unit 122A returns the processing to any of Step S210, Step S211, or Step S212, and calculates the recommended date and time again. The processing is repeated until the result of Step S250 becomes a YES determination.

As described above, according to the blood test support system 1, by outputting the performance of the postprandial blood collection to the medical examinee P, the postprandial hyperglycemia can be tested. Therefore, it is possible to detect diabetes early.

In a case where the after-a-meal is calculated as the recommended date and time, the following processing is preferably performed.

In a case where the after-a-meal is calculated as the recommended date and time, the server device 12 manages the acquisition of meal information from the medical examinee P. For example, in a case where the medical examinee P does not input and transmit information regarding meals to the terminal device 20 and the server device 12 does not acquire the meal information, it is possible to perform processing such as giving a warning not to perform self-blood collection, or not outputting the date-and-time-related information to the terminal device 20 of the medical examinee P even in a case where the recommended date and time approaches. However, in a case where the medical examinee P wears the blood glucose level sensor, the server device 12 can determine whether or not there is after a meal from the blood glucose level of the blood glucose level sensor. The medical examinee P does not need to input the information regarding the meal to the terminal device 20.

Additionally, in the case of the postprandial blood collection, in a case where 1.5 to 2 hours (meal registration is set to 0 hours) passes after a meal, there is a concern that it is not possible to accurately test whether or not the postprandial hyperglycemia is present even after blood collection. It is preferable to provide a notification in advance not to collect blood from 1.5 to 2 hours after a meal. Additionally, in a case where 1.5 to 2 hours have passed after a meal, it is preferable to display the next recommended date and time on the terminal device 20 of the medical examinee P.

In a case where the fasting time is calculated as the recommended date and time, the following processing is preferably performed. For example, it is preferable to issue a warning in a case where three hours or more have not passed after a meal. For example, in a case where the meal registration is set to 0 hour and in a case where the identification number of the blood test kit is read at the time of blood collection, if the time from the meal registration to the reading is within 3 hours, it is preferable to output a warning to the terminal device 20 of the medical examinee P.

Additionally, in a case where blood collection is performed at a fasting time and in a case where 10 o'clock AM is calculated as the recommended date and time for blood collection for the medical examinee P whose normal breakfast is 7 o'clock, it is preferable to change the recommended date and time to after 11:30 that is after the passage of 3 hours after a meal in a case where a breakfast is 8 o'clock (meal registration).

Additionally, in a case where the recommended date and time for the self-blood collection is output to the medical examinee P, it is preferable to output to the medical examinee P information on a post in which the blood test kit 100 is mailed. The information of the mailing post can include positional information of the mailing post, and collection and delivery date-and-time information. For example, information on the nearest post and collection and delivery time, and information at the fastest collection and delivery time at which mailing is possible and nearby posts can be presented on the basis of the address of the medical examinee P. The medical examinee P can perform mailing to any of the posts.

Moreover, the recommended date and time for the self-blood collection may be calculated in consideration of the collection and delivery time of a post to be mailed, and may be output to the medical examinee P. For example, in a case where the nearest collection and delivery time is 11:00 AM, 9:30 to 10:30 can be calculated as the recommended date and time for the self-blood collection.

Additionally, after the blood test kit 100 is mailed to a post, the medical examinee P may associate the mailing post with the identification number of the blood test kit 100 and perform transmission from the terminal device 20 to the server device 12. In a case where the server device 12 acquires the mailing information of the mailing post and the identification number, for example, the mailing information may be output to the test agency 30. Since the date and time at which the blood test kit 100 arrives at the test agency 30 can be predicted from the mailing information, the test agency 30 can predict the amount of work required for the test in advance.

It is preferable to calculate the recommended date and time except for a case where the blood-collection performance date or delivery period is at least one of Saturdays, Sundays, national holidays, long holiday seasons, or year-end and New Year holidays. This is for avoiding a state in which the blood test kit 100 is not sent to the test agency 30 after the blood test kit 100 is mailed, and for avoiding that the blood test kit 100 arrives at the test agency 30 during a period in which the test agency 30 does not perform the test work. This is for shortening the time from the date and time at which the medical examinee P performed self-blood collection to a test in the test agency 30. Here, the long holiday seasons mean four or more consecutive holidays in which Saturdays, Sundays, national holidays, and the like are consecutive. The blood test kit 100 remaining in the post for a few days after being mailed is avoided. Particularly, in the summer, there is a concern that the temperature inside the post becomes high and the blood sample of the blood test kit 100 deteriorates. It is preferable to calculate the recommended date and time in consideration of the number of days for which the blood sample stays in the post depending on the seasons. For example, the recommended date and time can be calculated based on the determination criteria that the number of days for which the blood test kit 100 stays in the post in summer is set to one day or less, the number of days for which the blood test kit 100 stays in the post in the spring and autumn is set to three days or less, and the number of days for which the blood test kit 100 stays in the post in the winter is set to 4 days or less. The number of days for each season can be appropriately changed.

Moreover, it is preferable to calculate the recommended date and time on the basis of at least one of a season, the outside temperature of an area, or the presence or absence of a cooling agent, in the place of residence of the medical examinee P. This is for reducing the influence of the outside temperature on the blood sample contained in blood test kit 100 and perform a highly accurate blood test, from the date and time at which self-blood collection is performed by the medical examinee P to a test in the test agency 30.

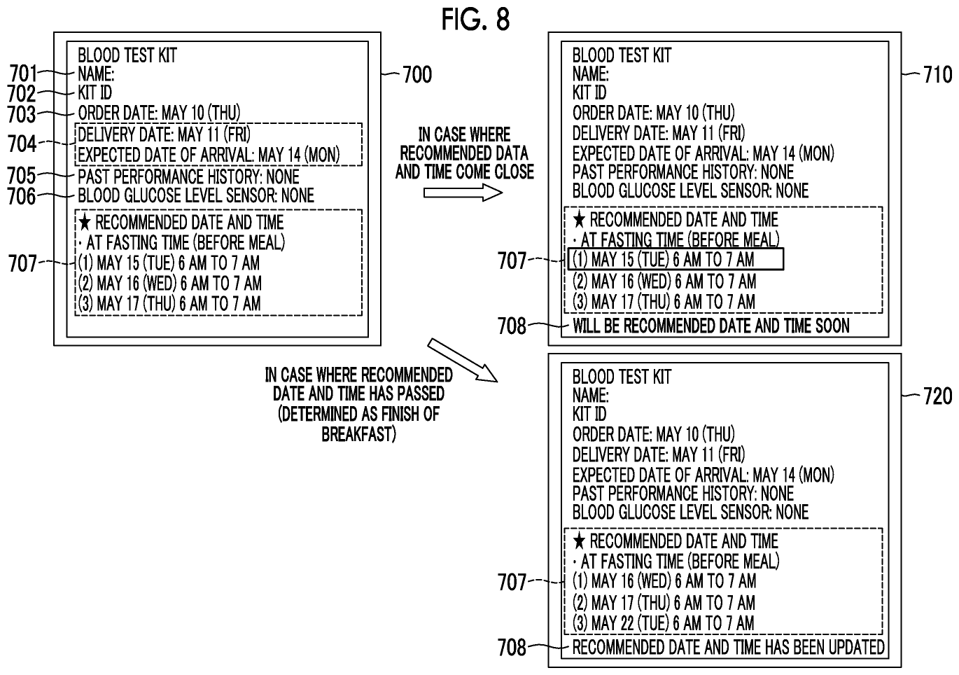

Next, regarding the recommended date and time, an example of a screen displayed on the terminal device 20 of the medical examinee P will be described with reference to FIGS. 8 to 10.

FIG. 8 is an example of a recommended date-and-time display screen in a case where there is no test performance history in the past and the blood glucose level sensor is not worn. The display screen 700 includes a display region 701 indicating the information of the medical examinee P, a display region 702 indicating the identification number of the blood test kit, a display region 703 indicating the information on the order, a display region 704 indicating the information on the delivery of the blood test kit, a display region 705 indicating the information on the past performance history, a display region 706 indicating the information on the wearing of the blood glucose level sensor, and a display region 707 displaying the information on the recommended date and time. In the embodiment, the display region 707 that displays the information on the recommended dates and times displays three recommended dates and times. In the display region 707, a fasting time (before-breakfast), (1) March 15 (Tuesday) 6 AM to 7 AM, (2) March 16 (Wednesday) 6 AM to 7 AM, and (3) March 3/17 (Thursday) 6 AM to 7 AM are calculated and displayed as the recommended dates and times. Since the recommended dates and times are displayed in the display region 707, it is not necessary for the medical examinee P to determine the date and time for self-blood collection, and the burden on the medical examinee P is reduced. However, the regions indicating the information included in the display screen 700 are not limited to these.

Next, a display screen 710 in a case where the recommended date and time is approaching will be described. In addition, in the display screen 710, some reference signs are omitted. In a case where the recommended date and time is approaching, the display screen 700 changes to the display screen 710. In the display screen 710, as illustrated in the figure the recommended date and time “March 15 (Tuesday) 6 AM to 7 AM” is displayed. Moreover, a display region 708 that displays the date-and-time-related information is provided adjacent to the display region 707. In the display region 708, the date-and-time-related information for prompting the medical examinee P to perform the self-blood collection, such as “will be a recommended date and time soon” output from the server device 12, is highlight-displayed. The burden on the medical examinee P regarding the performance date and time for the self-blood collection is reduced.

In a case where the identification number of the blood test kit 100 is transmitted from the terminal device 20 of the medical examinee P to the server device 12, the server device 12 determines that the medical examinee P has finished the self-blood collection by the blood test kit 100. The server device 12 finishes the management regarding the recommended date and time.

Next, the display screen 720 in a case where the recommended date and time has passed will be described. In addition, in the display screen 720, some reference signs are omitted. In a case where the recommended date and time has passed, the display screen 700 changes to the display screen 720. In the display screen 720, as illustrated in the figure, the recommended date and time is moved forward, and as the recommended dates and times, (1) March 16 (Wednesday) 6 AM to 7 AM and (2) March 17 (Thursday), 6 AM to 7 AM are calculated and displayed in the display region 707. As the third recommended date and time, (3) March 22 (Tuesday), 6 AM to 7 AM is calculated and displayed in the display region 707. In addition, March 22 (Tuesday) is calculated and displayed in consideration of the fact that March 18 is Friday and March 21 is a national holiday. In the display region 708, date-and-time-related information for notifying the medical examinee P of a change in the recommended date and time for the self-blood collection, such as “recommended date and time updated” output from the server device 12, is displayed. The burden on the medical examinee P regarding the performance date and time for the self-blood collection is reduced.

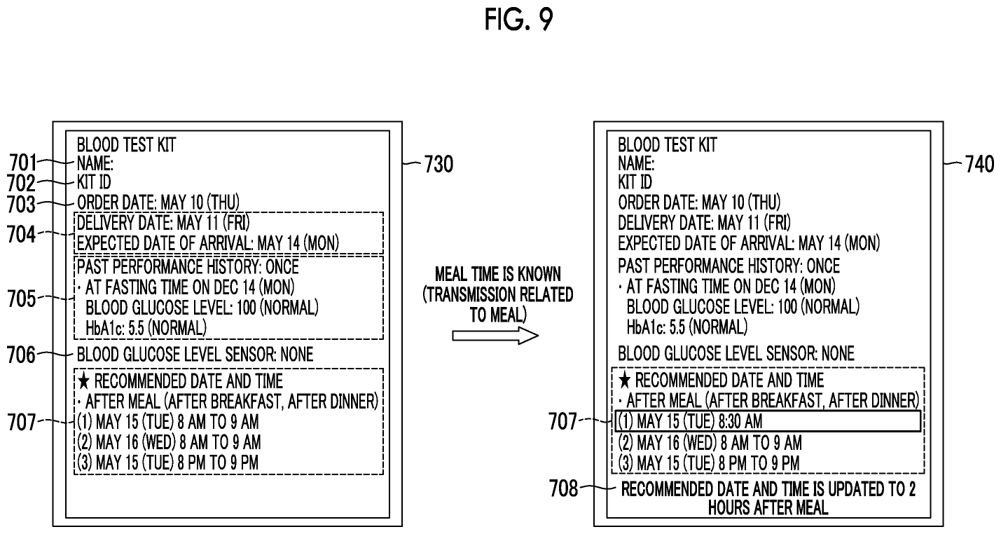

FIG. 9 is an example of the recommended date-and-time display screen in a case where there is a performance history of tests in the past and the blood glucose level sensor is not worn. There is a case where the same display regions as those in FIG. 8 are denoted by the same reference signs and the description thereof is omitted. The display region 705 of the display screen 730 includes information on a past test performance history. The display region 705 displays the past performance history: once, fasting blood glucose level on December 14 (Monday): 100 mg/dL (normal), and HbA1c: 5.5% (normal). In addition, the past test performance history may be displayed as a graph.

In the display region 707, as recommended dates and times, after-a-meal (after-breakfast or after-dinner), (1) March 15 (Tuesday) 8 AM to 9 AM, (2) March 16 (Wednesday) 8 AM to 9 AM, and (3) March 15 (Tue) 8 PM to 9 PM are calculated and displayed.

In a case where the information related to a meal is transmitted from the terminal device 20 of the medical examinee P, the server device 12 calculates the recommended date and time in consideration of the time of the meal. The server device 12 outputs the calculated recommended date and time to the terminal device 20 of the medical examinee P. The information related to the meal includes, for example, information on the time of the meal and the menu of the meal.

In a case where the information on the meal is transmitted, the display screen 730 is changed to the display screen 740. The recommended date and time is (1) March 15 (Tuesday), 8:30 AM, and is highlighted. Additionally, in the display region 708, date-and-time-related information for notifying the medical examinee P of a change in the recommended date and time for the self-blood collection, such as “the recommended date and time will be updated to 2 hours after a meal” output from the server device 12, is displayed. The burden on the medical examinee P regarding the performance date and time for the self-blood collection is reduced. In addition, in the display screen 740, some reference signs are omitted.

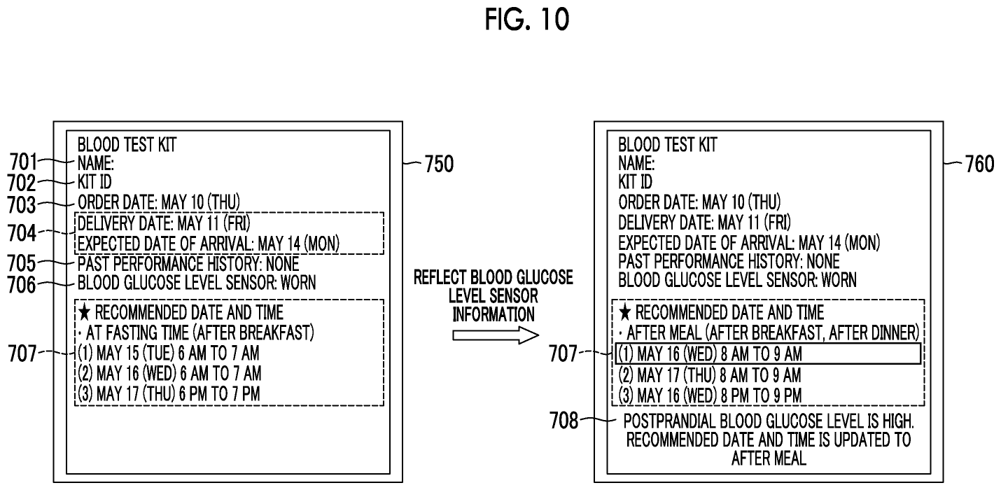

FIG. 10 is an example of the recommended date-and-time display screen in a case where there is no test performance history in the past and the blood glucose level sensor is worn. There is a case where the same display regions as those in FIG. 8 are denoted by the same reference signs and the description thereof is omitted. The display region 706 of a display screen 750 displays the wearing regarding the blood glucose level sensor.

In the display region 707, a fasting time (before-breakfast), (1) March 15 (Tuesday) 6 AM to 7 AM, (2) March 16 (Wednesday) 6 AM to 7 AM, and (3) March 3/17 (Thursday) 6 AM to 7 AM are calculated and displayed as the recommended dates and times.

In a case where the server device 12 receives the information indicating that the postprandial blood glucose level is high from the blood glucose level sensor, the server device 12 calculates the recommended date and time in consideration of the information of the blood glucose level sensor. The server device 12 outputs the recommended date and time to the terminal device 20 of the medical examinee P.

In a case where the blood glucose level sensor information is transmitted, the display screen 750 changes to a display screen 760. In the display region 707, after-a-meal (after-breakfast or after-dinner), (1) March 16 (Wednesday) 8 AM to 9 AM, (2) March 17 (Thursday) 8 AM to 9 AM, and (3) March 16 (Wednesday) 8 PM to 9 PM k is calculated and displayed as recommended dates and times. Additionally, in the display region 708, the date-and-time-related information for notifying the medical examinee P of a change in the recommended date and time for the self-blood collection, such as “the postprandial blood glucose level is high, the recommended date and time is updated after a meal” output from the server device 12, is displayed. The burden on the medical examinee P regarding the performance date and time for the self-blood collection is reduced. In addition, some reference signs are omitted in the display screen 760.

### Second Embodiment

[Overall Configuration of Blood Test Support System]

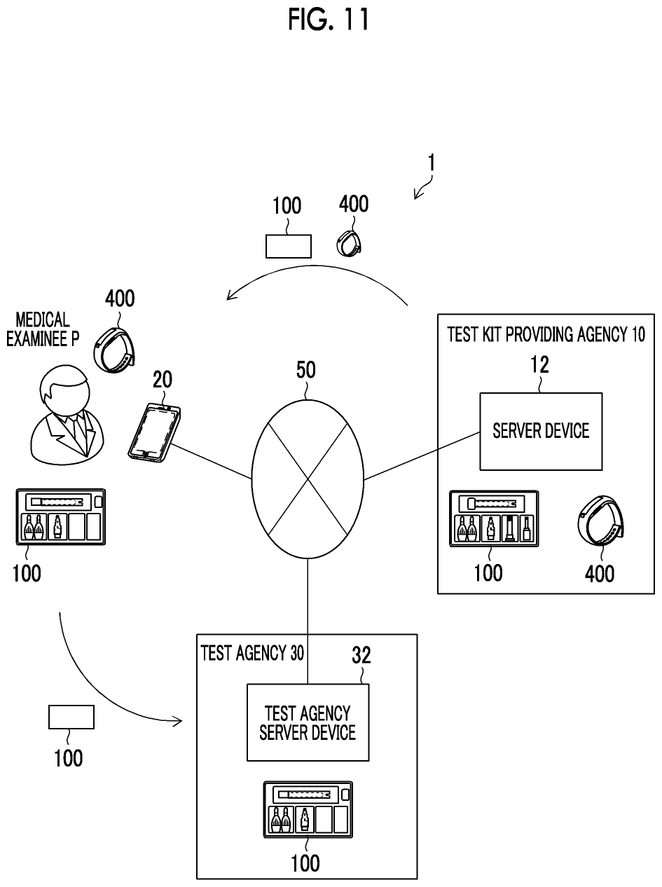

FIG. 11 is a diagram illustrating a configuration example of a blood test support system of the second embodiment. There is a case where the same components as those of the blood test support system of FIG. 1 are denoted by the same reference signs and the description thereof is omitted. The blood test support system 1 of the second embodiment prepares a lifestyle improvement plan on the basis of the result of a blood test of the medical examinee P, obtains approval from the medical examinee P, and transmits the lifestyle improvement plan to the medical examinee P. The blood test support system 1 of the second embodiment acquires body-related information related to the lifestyle improvement behavior of the medical examinee P, and calculates a recommended date and time at which blood collection is performed using the blood test kit to which the identification number is given, on the basis of the body-related information of the medical examinee P. The recommended date and time is transmitted to the medical examinee P.

Since the recommended date and time is calculated on the basis of body-related information related to lifestyle improvement behavior, the burden of managing the date and time for the self-blood collection can be reduced for the medical examinee P. Additionally, the medical examinee P can grasp the improvement situation according to the lifestyle improvement plan. Additionally, since the information on the blood test associated with the lifestyle improvement plan can be obtained, it is easy for the medical examinee P to have appropriate processing performed in consultation with the doctor on the basis of the information on the blood test.

As illustrated in FIG. 11, the blood test support system 1 comprises a server device 12 of a test kit providing agency 10 connected such that data communication is possible via a network 50, and a terminal device 20 owned by a medical examinee P. In addition, the server device 12 is equivalent to an example of a blood test support device.

In the embodiment illustrated in FIG. 11, the server device 12 is connected to the test agency server device 32 of the test agency 30 via the network 50.

Upon receiving a request from the medical examinee P, the test kit providing agency 10 provides the medical examinee P with a blood test kit 100 capable of performing self-blood collection. The test kit providing agency 10 prepares and transmits the lifestyle improvement plan to the medical examinee P who desires the lifestyle improvement behavior. Additionally, the test kit providing agency 10 also provides the biosensor 400 to the medical examinee P. The biosensor 400 monitors the body-related information of the medical examinee P, so that the server device 12 can acquire the body-related information related to the lifestyle improvement behavior performed by the medical examinee P.

The biosensor 400 is, for example, a disposable wearable device. The biosensor 400 can acquire biological information such as blood glucose level, heart rate, and respiratory rate, activity amount such as sleeping time, number of steps, travel distance, and the number of calories burned, and behavior information such as meal time. However, the biosensor is not limited to these. In addition, the biosensor 400 is an example for supporting the lifestyle improvement behavior of the medical examinee P. In order to support the lifestyle improvement behavior of the medical examinee P, the test kit providing agency 10 provides the medical examinee P with an application program for managing lifestyle improvement behaviors, a lifestyle improvement supplement, and the like in addition to the biosensor 400. The medical examinee P can apply any one or a combination of the biosensor 400, the application program, and the lifestyle improvement supplement. Those other than the above can also be provided to the medical examinee P in order to support lifestyle improvement behaviors.

The medical examinee P is a person who desires to improve his/her lifestyle by using the blood test kit 100, and is also referred to as a subject. The medical examinee P can order the blood test kit 100 from the terminal device 20 via the network 50 to the test kit providing agency 10 and can transmit his/her intention to desire the lifestyle improvement. The terminal device 20 may be, for example, a mobile terminal represented by a smartphone, a tablet, or the like or may be a personal computer.

The medical examinee P receives the blood test kit 100 and the biosensor 400 that are sent from the test kit providing agency 10. In a case where the server device 12 acquires the body-related information from the biosensor 400 of the medical examinee P, the server device 12 outputs the recommended date and time for the self-blood collection to the medical examinee P. In a case where the medical examinee P receives the recommended date and time, blood collection is performed using the blood test kit 100 according to the recommended date and time. The medical examinee P sends the blood test kit 100 for which blood collection has been completed to the test agency 30. The biosensor 400 may be held by the medical examinee P or may be discarded. Additionally, in a case where the medical examinee P already holds the biosensor 400, the biosensor 400 of the medical examinee P can transmit the body-related information to the server device 12.

The test agency 30 receives the blood test kit 100 sent from the medical examinee P. The test agency 30 tests the collected blood sample of the blood test kit 100 using a blood analysis device or the like. The test agency 30 acquires a numerical value for each test item for the blood sample. The test agency 30 can store the test result in the test agency server device 32 in association with the identification number of the blood test kit 100.

The test kit providing agency 10 can acquire the test result stored in the test agency server device 32 into the server device 12 via the network 50 and store the test result in association with the identification number of the blood test kit 100. Moreover, the server device 12 can store the body-related information of the medical examinee P in association with the identification number of the blood test kit 100. The medical examinee P can acquire the body-related information accompanying the lifestyle improvement and the test result via the terminal device 20.

The lifestyle improvement includes behaviors such as diet, exercising, drinking, and smoking, and all behaviors for maintaining and improving the health of the medical examinee P in relation to physical information such as weight and blood glucose level.

In FIG. 11, the test kit providing agency 10 and the test agency 30 are separate agencies, but may be an agency in which the test kit providing agency 10 and the test agency 30 are integrated with each other. In the case of the integrated agency, the server device 12 can comprise the function of the test agency server device 32.

The configuration of the blood test support system 1 of the second embodiment will be described. In the blood test support system 1 of the second embodiment, the server device 12 can apply the same hardware configuration as that in FIG. 2.

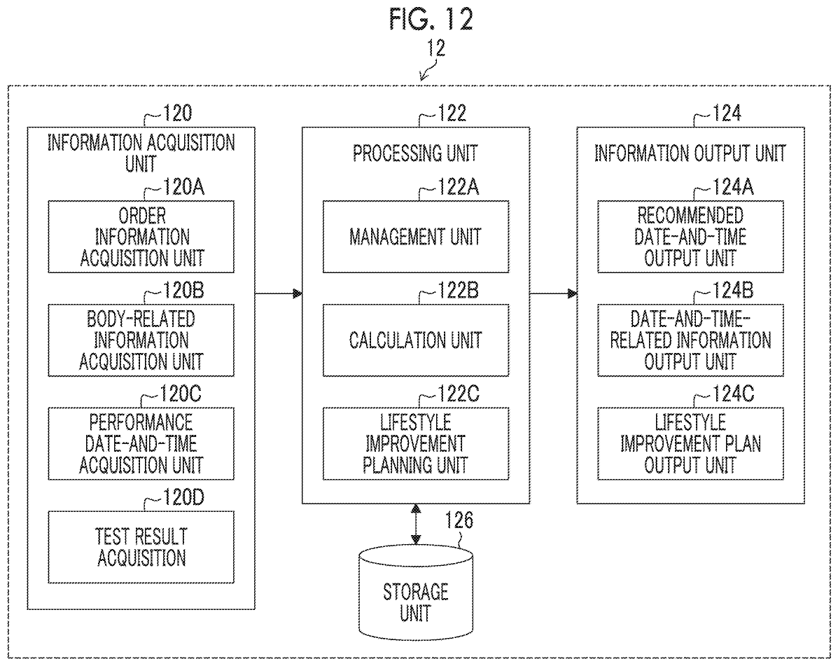

FIG. 12 is a functional block diagram of the server device 12. The server device 12 illustrated in FIG. 12 comprises an information acquisition unit 120, a processing unit 122, an information output unit 124, and a storage unit 126. There is a case where the same components as those of the server device 12 illustrated in FIG. 3 are denoted by the same reference signs and the description thereof is omitted.

The server device 12 illustrated in FIG. 12 comprises a body-related information acquisition unit 120B in the information acquisition unit 120, similarly to the server device 12 of FIG. 3.

The body-related information acquisition unit 120B acquires the body-related information of the medical examinee P. The body-related information includes information obtained by measurement, information reported by the medical examinee P, and information obtained by using various management application programs. In the second embodiment, the body-related information includes information related to the lifestyle improvement behavior of the medical examinee P. The body-related information is measured by an installed measuring device, a wearable measuring device such as a biosensor that the medical examinee P can wear, or the like. Examples of the body-related information include biological information such as height, weight, blood glucose level, and heart rate, and behavior information such as the number of exercises, the number of cigarettes smoked, the amount of alcohol drunk, the type and acquisition frequency of supplements, and meals. However, the type and acquisition method of the body-related information are not particularly limited.

The server device 12 illustrated in FIG. 12 comprises a lifestyle improvement planning unit 122C in the processing unit 122 in addition to the management unit 122A and the calculation unit 122B.

The lifestyle improvement planning unit 122C prepares a lifestyle improvement plan for the medical examinee P on the basis of the past test results. The lifestyle improvement plan includes a proposal regarding the behavior improvement such that numerical values required to be improved among test items obtained in a blood test fall within a predetermined numerical range. The behavior improvement includes, for example, dietary conditions, exercising conditions, drinking conditions, smoking conditions, and supplement intake conditions. The behavior improvement presented to the medical examinee P is prepared, for example, on the basis of behavior improvement information regarding the behavior improvement obtained from past cases or the like. The behavior improvement information may be information stored in the storage unit 126 or information stored in a storage device such as an external database.

The server device 12 illustrated in FIG. 12 comprises a lifestyle improvement plan unit output unit 124C in the information output unit 124, in addition to the recommended date-and-time output unit 124A and the date-and-time-related information output unit 124B.

In the blood test support system 1 of the second embodiment, the terminal device 20 can provide the same hardware configuration as that in FIG. 4. Therefore, the terminal device 20 can download various application programs via the network 50 and can execute the downloaded various application programs.

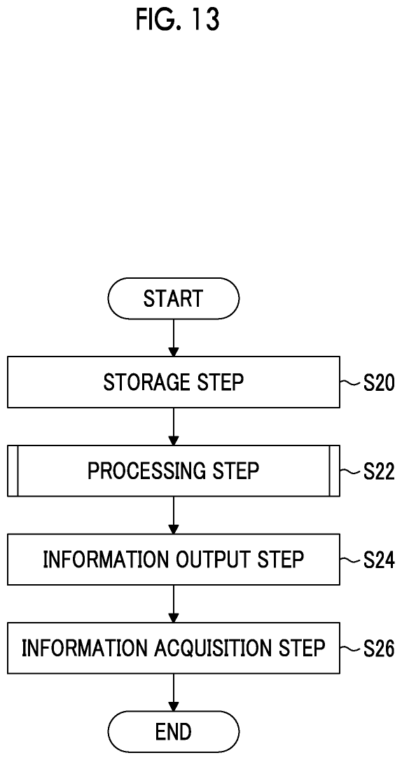

Next, a flow of a blood test support method of the second embodiment will be described. As illustrated in FIG. 13, there is provided a blood test support method including a storage step (Step S20) of associating and storing the medical examinee P and a test result of the medical examinee P; a processing step (Step S22) of preparing a lifestyle improvement plan for the medical examinee P on the basis of the test result; an information output step (Step S24) of outputting the lifestyle improvement plan to the medical examinee P; an information acquisition step (Step S26) of acquiring an order for the blood test kit 100 from the medical examinee P, in the information acquisition step (Step S24), body-related information of the medical examinee P and an identification number given to the blood test kit are acquired, in the storage step (Step S20), the body-related information and the identification number are stored in association with each other, in the processing step (Step S22), a recommended date and time at which blood collection is performed using the blood test kit 100 to which the identification number is given is calculated on the basis of the body-related information of the medical examinee P, and in the information output step (Step S24), the identification number of the blood test kit 100 and the recommended date and time are output to the medical examinee P.

The above-described blood test support method can be configured as a program that causes a computer to realize functions corresponding to the respective units in the blood test support device and functions corresponding to the respective steps in the blood test support method.

For example, a blood test support program that causes a computer to realize a storage function, a processing function, an information output function, and an information acquisition function may be configured.

It is possible to store the program of causing the program to realize the above-described blood test support functions in a computer-readable information storage medium that is a non-temporary information storage medium that is a tangible object, and provide the program through the information storage medium.

Additionally, instead of storing the program in the non-temporary information storage medium to provide the program, an aspect in which the program signal is provided via a network is also possible.

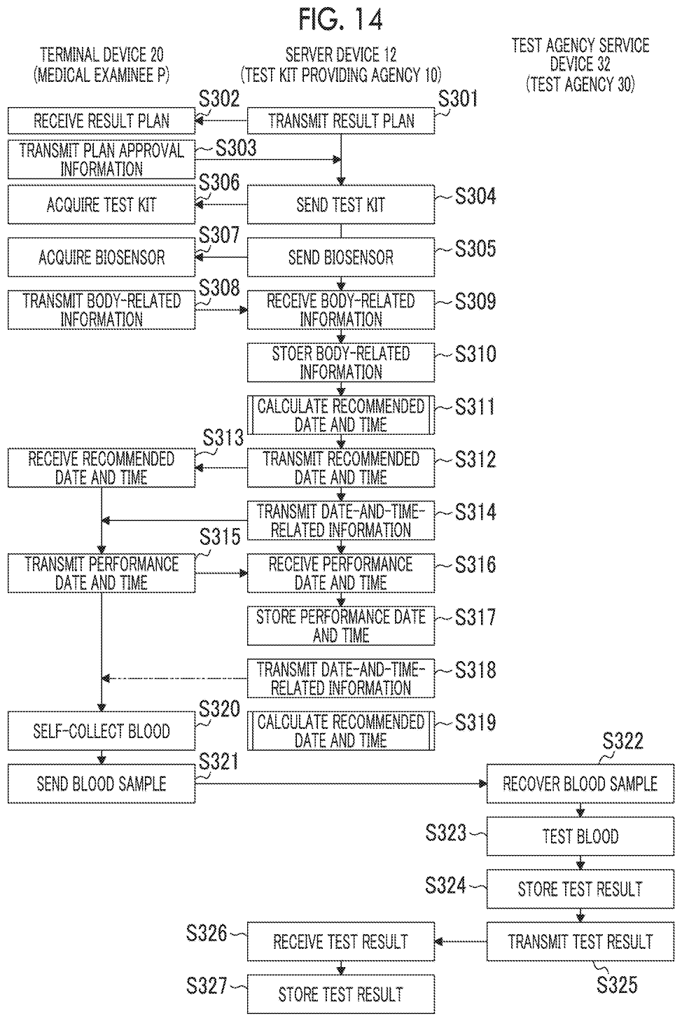

FIG. 14 is a diagram illustrating a flow of information and a flow of the blood test kit and the biosensor in the blood support system of the second embodiment. In the second embodiment, the medical examinee P performs a blood test using the blood test kit 100 at least once, and prepares a lifestyle improvement plan on the basis of the test result. Therefore, regarding the first blood test, the flow of information and the flow of the blood test kit in the blood support system are similar to those in FIG. 6. Therefore, FIG. 14 illustrates the flow of information and the flow of the blood test kit in the blood support system after the server device 12 acquires the result of the blood test.

In a result plan transmission step S301, the result of the blood test and the lifestyle improvement plan are transmitted from the server device 12 to the terminal device 20 of the medical examinee P. The lifestyle improvement plan is prepared by the lifestyle improvement planning unit 122C of the server device 12, and the result of the blood test and the lifestyle improvement plan are output from the lifestyle improvement plan output unit 124C to the medical examinee P.

In a result plan reception step S302, the terminal device 20 receives the result of the blood test and the lifestyle improvement plan that are transmitted from the server device 12. In a case where the medical examinee P improves his/her behavior according to the lifestyle improvement plan and desires a retest using the blood test kit 100, in a plan approval information transmission step S303, approval information indicating that the plan is approved is transmitted from the terminal device 20 to the server device 12.

In a test kit sending step S304, the test kit providing agency 10 sends the blood test kit 100 to the medical examinee P on the basis of the approval information, and in a biosensor sending step S305, the test kit providing agency 10 sends the biosensor 400 to the medical examinee P on the basis of the approval information. The biosensor 400 is for supporting the lifestyle improvement plan of the medical examinee P, and is not limited to the biosensor 400 and may be a supplement or an application program that executes various types of management. Examples of the application program include at least one of a diet calorie calculation application program, a smoking management application program, or a drinking amount management application program. The diet calorie calculation application program can automatically calculate diet calories and nutrients from, for example, a captured image, and can set the time at which the image has been captured as meal intake time. The smoking management application program can start the application program, for example, every smoking and can count the number of cigarettes, for example, by tapping a button. The drinking amount management application program can manage the drinking amount by inputting the number and the type of each drinking.

In a test kit acquisition step S306 and a biosensor kit acquisition step S307, the medical examinee P acquires the blood test kit 100 and the biosensor 400 sent from the test kit providing agency 10, respectively.

In a body-related information transmission step S308, the information related to the lifestyle improvement behavior of the medical examinee P is transmitted from the terminal device 20 and the biosensor 400 to the server device 12. In a body-related information reception step S309, the server device 12 acquires the information related to the lifestyle improvement behavior. In a body-related information storage step S310, the body-related information is stored in the storage unit 126 of the server device 12 in association with the identification number of the blood test kit 100.

After the body-related information storage step S310, the processing proceeds to a recommended date-and-time calculation step S311. In the recommended date-and-time calculation step S311, the calculation unit 122B of the processing unit 122 calculates a recommended date and time on the basis of the lifestyle improvement plan and the body-related information of the medical examinee P.

In a recommended date-and-time transmission step S312, the server device 12 transmits the recommended date and time and the identification number of the blood test kit 100 to the terminal device 20. In the recommended date-and-time transmission step S312, the recommended date and time is transmitted to the terminal device 20 of the medical examinee P via the recommended date-and-time output unit 124A. The management unit 122A may transmit information, indicating that the recommended date and time has been calculated, to the terminal device 20 via the information output unit 124. For example, the management unit 122A can transmit information, indicating that the recommended date and time has been calculated, to the terminal device 20 using an e-mail, an alert, or the like.

In a recommended date-and-time reception step S313, the terminal device 20 receives the recommended date and time transmitted from the server device 12. The medical examinee P can confirm the recommended date and time via the display device 254 of the terminal device 20.

In a date-and-time-related information transmission step S314, the management unit 122A transmits information related to the recommended date and time to the terminal device 20 of the medical examinee P via the date-and-time-related information output unit 124B.

In a performance date-and-time transmission step S315, a performance date and time is transmitted from the terminal device 20 of the medical examinee P to the server device 12. The medical examinee P can input the performance date and time from the input device 256 of the terminal device 20. The medical examinee P can input the identification number of the blood test kit 100 by the camera unit 258 of the terminal device 20 and input the imaged date and time to the terminal device 20 as the performance date and time.

In a performance date-and-time reception step S316, the server device 12 receives the performance date and time transmitted from the terminal device 20. In a performance date-and-time storage step S317, management unit 122A stores the performance date and time in the storage unit 126 in association with the identification number of the blood test kit 100.

In addition, in a case where the performance date and time and the recommended date and time are different from each other, in a date-and-time-related information transmission step S318, warning information is transmitted to the terminal device 20 of the medical examinee P. In a case where the performance date and time and the recommended date and time match each other, the date-and-time-related information transmission step S318 is not performed.

As necessary, there is a case where the recommended date-and-time calculation step S319 is executed to calculate a new recommended date and time. The management unit 122A manages the new recommended date and time.

In a self-blood collecting step S320, the medical examinee P performs self-blood collection using the blood test kit 100 according to the recommended date and time. In a blood sample sending step S321, the medical examinee P sends the blood test kit 100 in which a blood sample is collected, to the test agency 30.

In the blood sample recovering step S322, the test agency 30 recovers the blood test kit 100 including the blood sample of the medical examinee P sent from the medical examinee P. In a blood testing step S323, the test agency 30 tests the blood sample of the medical examinee P. In a test result storage step S324, a test result is stored in a storage unit of the test agency server device 32 in association with the medical examinee P and the identification number of the blood test kit 100. In a test result transmission step S325, a blood test of the medical examinee P stored in the test agency server device 32 is transmitted to the server device 12.

In the test result reception step S326, the test result stored in the test agency server device 32 is acquired. The test result is associated with the identification number of blood test kit 100. In the test result storage step S327, the test result obtained in the test result reception step S122 is stored in the server device 12. The management unit 122A of the processing unit 122 stores the test result and the identification number of the blood test kit 100 in the storage unit 126 in association with each other.

The medical examinee P can browse the body-related information accompanying the lifestyle improvement and the test result in association with each other.

Next, the case of Pattern 1 in which the medical examinee P performs a blood test after performing a normal blood test for the first time and performs a blood test after performing a lifestyle improvement behavior for the second time will be described.

The medical examinee P transmits order information to the server device 12 of the test kit providing agency 10 via the terminal device 20. On the basis of the order information, the blood test kit 100 is sent from the test kit providing agency 10 to the medical examinee P.

The medical examinee P transmits an identification number of the blood test kit 100 from the terminal device 20 to the server device 12. The server device 12 stores a date and time at which the information has been acquired, as the performance date and time in association with the identification number. The medical examinee P sends the blood test kit 100 to the test agency 30 after the self-blood collection. The test agency 30 tests the blood sample of the blood test kit 100 and transmits a test result to the server device 12.

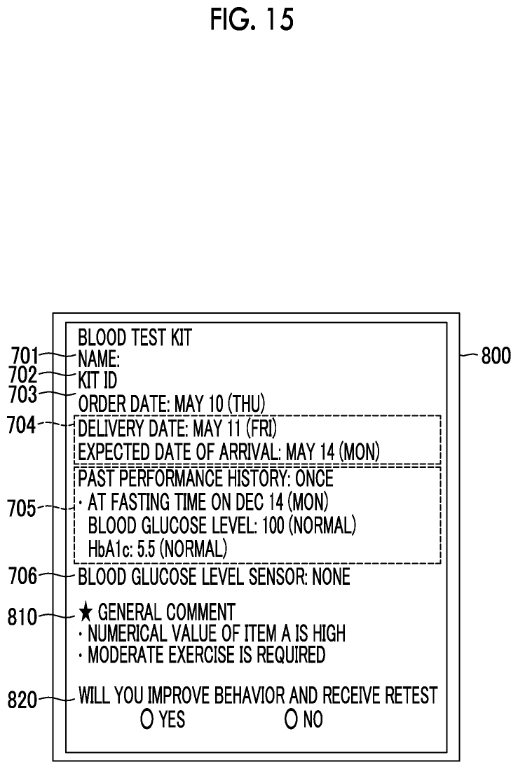

Next, the test result, and the lifestyle improvement plan based on the test result are output from the server device 12 to the terminal device 20 of the medical examinee P. For example, a screen illustrated in FIG. 15 is displayed on the terminal device 20. The display screen 800 includes the display region 701 indicating the information of the medical examinee P, the display region 702 indicating the identification number of the blood test kit, the display region 703 indicating the information on the order, the display region 704 indicating the information on the delivery of the blood test kit, the display region 705 indicating the information on the past performance history, and the display region 706 indicating the information on the wearing of the blood glucose level sensor.

Moreover, the display screen 800 includes a display region 810 indicating a general comment and a display region 820 indicating an approval of a lifestyle improvement plan. In the display region 810, an item of a test result to be improved and a lifestyle improvement plan to be improved (here, suggestion of moderate exercise) are displayed.

In the display region 820, a display of “Do you want to improve your behavior and receive a retest?” and a selection button that allows you to choose “Yes” or “No” can be displayed.

Next, in a case where the medical examinee P selects “Yes”, plan approval information is transmitted from the terminal device 20 to the server device 12.

Next, the biosensor 400, an application program for downloading, support products for supporting lifestyle improvement such as lifestyle improvement supplements (for example, for 30 days), and the blood test kit 100 are sent from the test kit providing agency 10 to the medical examinee P.

For example, in a case where the blood glucose level is high, the biosensor 400 that monitors the blood glucose level is sent, and in a case where bad cholesterol is high, the biosensor 400 that also monitors the heart rate and the respiratory state is sent. Additionally, in a case where a diet is improved, since the calories are automatically calculated, information on a link destination for a dietary management application program is sent to the medical examinee P. Additionally, supplements to improve the intestinal environment will be sent. The biosensor 400 is preferably a designated one. This is because a correlation is derived from a lot of acquired body-related information. For example, the medical examinee P can transmit behavior information such as aerobic exercise and meals to the server device 12 via the terminal device 20. It is preferable that the behavior information can be acquired by the biosensor 400. It is possible to reduce the burden on the medical examinee P of inputting information to the terminal device 20.

Next, as the medical examinee P wears the biosensor 400, the body-related information of the medical examinee P can be consecutively transmitted to the server device 12. The information acquired by the biosensor 400 can be wirelessly transmitted to the terminal device 20.

The server device 12 transmits the recommended date and time for the self-blood collection to the terminal device 20 of the medical examinee P on the basis of the body-related information. For example, in a case where it is determined on the basis of a monitoring result from the biosensor 400 that a behavior improvement has become a habit according to a lifestyle improvement plan, the server device 12 determines that the timing at which a blood test is performed has been reached, and outputs a recommended date and time for blood collection to the terminal device 20 of the medical examinee P. Whether or not the behavior improvement has become a habit can be determined that, for example, in a case where the target number of times described in the lifestyle improvement plan has been achieved, the behavior improvement has become a habit.

Hereinafter, examples of indicators that are targets of lifestyle improvement plans are shown. However, indicator items and numerical values can be different for each medical examinee P.

In relation to meals, an achievement in which meals after 10:00 PM are 5 days or less in the last one month and regular meals of 3 meals a day is 80% in the last one month, an achievement in which no skipping breakfast is 80% in the last one month, an achievement in which a daily vegetable intake amount of 350 g or more (target) is 80% in the last one month, an achievement in which a daily salt intake amount of 8 g or less (target) is 80% in the last one month, an achievement in which a daily calorie intake amount equal to or more than basic metabolism and equal to or less than a target value (calculated depending on height, weight, age, or the like) is 80% in the most recent month, and the like can be used as the indicators.

The target achievements can be determined by acquiring images of meals with the terminal device 20 of the medical examinee P and automatically calculating calories. Even in a case where a target regarding meals is not achieved, in a case where an improvement is seen in the blood glucose level obtained by the biosensor 400, the recommended date and time for blood collection may be output to the terminal device 20 of the medical examinee P.

In relation to the exercise, an achievement in which aerobic exercise such as walking is taken equal to or more than 3 days a week for 30 minutes in the last one month, an achievement in which, at a slightly higher level, the aerobic exercise is taken 3 days or more for a total of 150 minutes a week in the last one month, an achievement in which a daily average number of steps of 9000 (target for men over 20 years old) is 80% in the last one month, and the like can be used as the indicators. It is preferable to determine the target achievements from a respiratory state monitor by the biosensor 400. An input burden on the medical examinee P can be reduced.

In relation to other improvement behaviors, an achievement in which days to give one's liver a rest are equal to or more than three days a week in the last one month, an achievement of no smoking in the last one month, an achievement in which a sleeping time of 6 hours or more is 80% in the last one month, an achievement in which the number of days of supplements used is 80% or more in the last one month, and the like can be used as the indicators.

The above-described numerical values are examples, and can be appropriately changed depending on the test result of the medical examinee P.

Next, the medical examinee P performs the self-blood collection using the blood test kit 100 at the recommended date and time, and sends the blood test kit 100 to the test agency 30. A blood test is performed in the test agency 30, and the test result is transmitted to the server device 12.

Next, after the elapse of several days, the result of comparison between the present test result and the previous test result is output from the server device 12 to the terminal device 20 of the medical examinee P. The medical examinee P can easily grasp the relation between the lifestyle improvement behavior and the effect of the behavior.

Next, a screen similar to that illustrated in FIG. 15 is displayed on the terminal device 20 of the medical examinee P. A display of “Do you want to improve your behavior and receive a retest?” and a selection button that allows you to choose “Yes” or “No” can be displayed in the terminal device 20.

In a case where the medical examinee P selects “Yes”, the same procedure is repeated. The test result, and support products that support a so-called lifestyle improvement corresponding to a behavior improvement plan are sent from the test kit providing agency 10 to the medical examinee P.

Next, the case of Pattern 2 in which the medical examinee P performs a blood test after acquiring the body-related information for the first time and performs a blood test after performing a lifestyle improvement behavior for the second time will be described.

The medical examinee P transmits order information to the server device 12 of the test kit providing agency 10 via the terminal device 20. The blood test kit 100 is sent from the test kit providing agency 10 on the basis of the order information.

In a case where the medical examinee P makes an order via the terminal device 20, the medical examinee P transmits a message to the server device 12 that he/she wants to present his/her lifestyle improvement after monitoring usual body-related information.

Next, on the basis of the order information, the blood test kit 100 and the support products such as the biosensor 400 for supporting lifestyle improvement are sent from the test kit providing agency 10 to the medical examinee P. The medical examinee P wears the biosensor 400 and performs normal behaviors. The medical examinee P wears the biosensor 400 and performs the self-blood collection after 2-3 days.

The medical examinee P transmits an identification number of the blood test kit 100 from the terminal device 20 to the server device 12. The server device 12 stores a date and time at which the information has been acquired, as the performance date and time in association with the identification number. The medical examinee P sends the blood test kit 100 to the test agency 30 after the self-blood collection. The test agency 30 tests the blood sample of the blood test kit 100 and transmits a test result to the server device 12.

Next, the test result, and the lifestyle improvement plan based on the test result are output from the server device 12 to the terminal device 20 of the medical examinee P. In a case where the medical examinee P selects “Yes” in response to the display “Do you want to improve your behavior and receive a retest?”, the plan approval information is transmitted from the terminal device 20 to the server device 12.

Next, the biosensor 400, an application program for downloading, support products for supporting lifestyle improvement such as lifestyle improvement supplements (for example, for 30 days), and the blood test kit 100 are sent from the test kit providing agency 10 to the medical examinee P.

The server device 12 transmits the recommended date and time for the self-blood collection to the terminal device 20 of the medical examinee P on the basis of the body-related information. For example, in a case where it is determined on the basis of a monitoring result from the biosensor 400 that a behavior improvement has become a habit according to a lifestyle improvement plan, the server device 12 determines that the timing at which a blood test is performed has been reached, and outputs a recommended date and time for blood collection to the terminal device 20 of the medical examinee P.

Next, the medical examinee P performs the self-blood collection using the blood test kit 100 at the recommended date and time, and sends the blood test kit 100 to the test agency 30. A blood test is performed in the test agency 30, and the test result is transmitted to the server device 12.

Next, after the elapse of several days, the result of comparison between the present test result and the previous test result is output from the server device 12 to the terminal device 20 of the medical examinee P. The medical examinee P can easily grasp the relation between the lifestyle improvement behavior and the effect of the behavior.

Next, a screen similar to that illustrated in FIG. 15 is displayed on the terminal device 20 of the medical examinee P. A display of “Do you want to improve your behavior and receive a retest?” and a selection button that allows you to choose “Yes” or “No” can be displayed in the terminal device 20.

In a case where the medical examinee P selects “Yes”, the same procedure is repeated. The test result, and support products that support a so-called lifestyle improvement corresponding to a behavior improvement plan are sent from the test kit providing agency 10 to the medical examinee P.

In the case of Pattern 1 and Pattern 2, the medical examinee P can place an order for at least two sets of blood test kits 100 and at least one set of support products for supporting lifestyle improvement to the server device 12 via the terminal device 20 of the medical examinee P.

Similarly to the first embodiment, it is preferable to display the information of the mailing post on the terminal device 20 of the medical examinee P. Additionally, after the blood test kit 100 is mailed to a post, the medical examinee P may associate the mailing post with the identification number of the blood test kit 100 and perform transmission from the terminal device 20 to the server device 12.

In the second embodiment, on the basis of the previous blood test results, recommended items for diet and exercise behavior improvement can be extracted in the lifestyle improvement plan for the medical examinee P (for example, an achievement in which no skipping breakfast is 80% in the last one month, an achievement in which a daily average number of steps of 9000 (target for men over 20 years old) is 80% in the last one month, and the like, and the medical examinee P can select an item he/she intends to perform from these items (can select a plurality of items). Then, it is preferable that a display for achieving the behavior improvement items such as for example, “Pease eat breakfast” and “Walk 5000 steps today”, and “Walk a little more” are made on are displayed on the terminal device 20 of the medical examinee P on the basis of the selected lifestyle improvement plan.

Additionally, it is preferable that the performance situation is visualized in the selected behavior improvement item. The medical examinee P is encouraged toward achievement by being visualized. An application program that acquires life log information can also be used for behavior improvement support.

### EXPLANATION OF REFERENCES

10: test kit providing agency
12: server device
20: terminal device
30: test agency
32: test agency server device
50: network
100: blood test kit
112A: management unit
120: information acquisition unit
120A: order information acquisition unit
120B: body-related information acquisition unit
120C: performance date-and-time acquisition unit
120D: test result acquisition unit
122: processing unit
122A: management unit
122B: calculation unit
122C: lifestyle improvement planning unit
124: information output unit
124A: recommended date-and-time output unit
124B: date-and-time-related information output unit
124C: lifestyle improvement plan output unit
126: storage unit
140: control unit
142: memory
144: storage device
146: network controller
148: power supply device
152: bus
240: control unit
242: memory
244: storage device
246: network controller
248: power supply device
252: bus
254: display device
256: input device
258: camera unit
400: biosensor
700: display screen
701: display region
702: display region
703: display region
704: display region
705: display region
706: display region
707: display region
708: display region
710: display screen
720: display screen
730: display screen
740: display screen
750: display screen
760: display screen
800: display screen
810: display region
820: display region

1. A blood test support device comprising:
a storage configured to store an identification number given to a blood test kit; and
at least one processor configured to:
acquire an order for a blood test kit from a medical examinee;
store an identification number given to the blood test kit in the storage in association with the medical examinee;
calculate a recommended date and time at which blood collection is performed using the blood test kit to which the identification number is given; and
output the identification number of the blood test kit and the recommended date and time in placing the order from the medical examinee.

2. The blood test support device according to claim 1,
wherein the at least one processor calculates the recommended date and time except a case where a blood-collection performance date or delivery period is at least one of Saturday, Sunday, a national holiday, a long holiday season, or year-end and New Year holidays.

3. The blood test support device according to claim 1,
wherein the at least one processor calculates the recommended date and time on the basis of at least one of a season, an outside temperature of an area, or a presence or absence of a cooling agent, in a place of residence of the medical examinee.

4. The blood test support device according to claim 1,
wherein the at least one processor acquires a performance date and time for blood collection from the medical examinee, and stores the performance date and time and the identification number in association with each other in the storage.

5. The blood test support device according to claim 4,
wherein in a case where it is determined that the performance date and time and the recommended date and time are different from each other, the at least one processor outputs warning information.

6. The blood test support device according to claim 4,
wherein the at least one processor acquires a test result of the blood test kit to which the identification number is given, and stores the identification number, the performance date and time and the test result in association with the medical examinee in the storage.

7. The blood test support device according to claim 6,
wherein in a case where the at least one processor acquires the order for the blood test kit from the medical examinee, the at least one processor calculates the recommended date and time on the basis of the performance date and time of the medical examinee and the test result stored in the storage.

8. The blood test support device according to claim 1,
wherein a date and time at which early morning, fasting, and rest are predicted is calculated as the recommended date and time.

9. The blood test support device according to claim 7,
wherein the at least one processor calculates a self-blood collection after a meal as the recommended date and time in a case where the performance date and time is a fasting time and a blood glucose level and an HbA1c are normal.

10. The blood test support device according to claim 7,
wherein the at least one processor
calculates a self-blood collection after a meal as the recommended date and time in a case where the performance date and time is only a fasting time and a blood glucose level and an HbA1c are normal,
calculates the fasting time as the recommended date and time in a case where the performance date and time is not the fasting time or an unknown time, and the blood glucose level and the HbA1c are normal, and
calculates a performance date and time for blood collection in a case where the blood glucose level and the HbA1c are high as the recommended date and time, in a case where the performance date and time is not the fasting time and or an unknown time and the blood glucose level and the HbA1c are high either at the fasting time or after the meal, and calculates the fasting time as the recommended date and time in a case where the blood glucose level and the HbA1c are high both at the fasting time and after the meal and the after-the-meal as the recommended date and time in a case where the performance date and time is an unknown time.

11. A blood test support system comprising a server device configured to be connectable to a terminal device of a medical examinee via a network,
wherein the server device includes:
a storage configured to store an identification number given to a blood test kit; and
at least one processor configured to:
acquire an order for a blood test kit from a medical examinee;
store an identification number given to the blood test kit in the storage in association with the medical examinee;
calculate a recommended date and time at which blood collection is performed using the blood test kit to which the identification number is given; and
output the identification number of the blood test kit and the recommended date and time in placing the order from the medical examinee.

12. A blood test support method comprising:
an information acquisition step of acquiring an order for a blood test kit from a medical examinee;
a storage step of storing an identification number given to the blood test kit in association with the medical examinee;
a processing step of calculating a recommended date and time at which blood collection is performed using the blood test kit to which the identification number is given; and
an information output step of outputting the identification number of the blood test kit and the recommended date and time in placing the order from the medical examinee.

13. A blood test support device comprising:
a storage configured to store a test result of a medical examinee in association with the medical examinee; and
at least one processor configured to:
prepare a lifestyle improvement plan for the medical examinee on the basis of the test result
output the lifestyle improvement plan to the medical examinee; and
acquire an order for a blood test kit from the medical examinee,
wherein the at least one processor acquires body-related information of the medical examinee and an identification number given to the blood test kit,
stores the body-related information and the identification number in association with each other in the storage,
calculates a recommended date and time at which blood collection is performed using the blood test kit to which the identification number is given, on the basis of the body-related information of the medical examinee, and
outputs the identification number of the blood test kit and the recommended date and time to the medical examinee.

14. The blood test support device according to claim 13,
wherein the lifestyle improvement plan includes information on any of diet, exercise, smoking, or supplements.

15. A blood test support system comprising a server device configured to be connectable to a terminal device of a medical examinee via a network,
wherein the server device includes:
a storage configured to store a test result of a medical examinee in association the medical examinee; and
at least one processor configured to:
prepare a lifestyle improvement plan for the medical examinee on the basis of the test result;
output the lifestyle improvement plan to the medical examinee; and
acquire an order for a blood test kit from the medical examinee,
wherein the at least one processor acquires body-related information and an identification number given to the blood test kit,
stores the body-related information and the identification number in association with each other in the storage,
calculates a recommended date and time at which blood collection is performed using the blood test kit to which the identification number is given, on the basis of the body-related information of the medical examinee, and
outputs the identification number of the blood test kit and the recommended date and time to the medical examinee.

16. The blood test support system according to claim 15,
wherein the at least one processor acquires the body-related information via a terminal device owned by the medical examinee.

17. The blood test support system according to claim 16,
wherein the terminal device includes at least one of a mobile terminal or a biosensor.

18. The blood test support system according to claim 17,
wherein the mobile terminal includes at least one of a diet calorie calculation application program, a smoking management application program, or a drinking amount management application program.

19. The blood test support system according to claim 17,
wherein the biosensor is capable of acquiring information on any of an activity amount, an electrocardiogram, a heart rate, a heartbeat fluctuation, a respiratory state, a body surface temperature, a sleep analysis, a blood glucose level, or a blood oxygen concentration of the medical examinee.

20. A blood test support method comprising:
a storage step of storing a test result of a medical examinee in association the medical examinee;
a processing step of preparing a lifestyle improvement plan for the medical examinee on the basis of the test result;
an information output step of outputting the lifestyle improvement plan to the medical examinee; and
an information acquisition step of acquiring an order for a blood test kit from the medical examinee,
wherein in the information acquisition step, the body-related information of the medical examinee and an identification number given to the blood test kit are acquired,
wherein in the storage step, the body-related information and the identification number are stored in association with each other,
wherein in the processing step, a recommended date and time at which blood collection is performed using the blood test kit to which the identification number is given is calculated on the basis of the body-related information of the medical examinee, and
wherein in the information output step, the identification number of the blood test kit and the recommended date and time are output to the medical examinee.

21. A non-temporary and computer-readable recording medium, in a case where a command stored in the recording medium are read by a computer, for causing a computer to realize a blood test support function in a case where a command stored in the recording medium is read by the computer, the blood test support function comprising:
an information acquisition function of acquiring an order for a blood test kit from a medical examinee;
a storage function of storing an identification number given to the blood test kit in association with the medical examinee;
a processing function of calculating a recommended date and time at which blood collection is performed using the blood test kit to which an identification number is given; and
an information output function of outputting the identification number of the blood test kit and the recommended date and time at which an order is placed from the medical examinee.

22. A non-temporary and computer-readable recording medium, in a case where a command stored in the recording medium are read by a computer, for causing a computer to realize a blood test support function in a case where a command stored in the recording medium is read by the computer, the blood test support function comprising:
a storage function of storing a test result of a medical examinee in association with the medical examinee;
a processing function of preparing a lifestyle improvement plan for the medical examinee on the basis of the test result;
an information output function of outputting the lifestyle improvement plan to the medical examinee; and
an information acquisition function of acquiring an order for a blood test kit from the medical examinee,
wherein in the information acquisition function, the body-related information of the medical examinee and an identification number given to the blood test kit are acquired,
wherein in the storage function, the body-related information and the identification number are stored in association with each other,
wherein in the processing function, a recommended date and time at which blood collection is performed using the blood test kit to which the identification number is given is calculated on the basis of the body-related information of the medical examinee, and
wherein in the information output function, the identification number of the blood test kit and the recommended date and time are output to the medical examinee.
